(19) United States
(12) Patent Application Publication
(10) Pub. No.: US 2026/0317269 A1
(43) Pub. Date: Oct. 15, 2026

(54) Oxamate Derivatives and Prodrugs to Prevent CaOx Stone Formation

(71) Applicant: Rutgers, The State University of New Jersey, New Brunswick, NJ (US)

(72) Inventors: Longqin Hu, Belle Mead, NJ (US); Yiling Wang, Shanghai (CN)

(21) Appl. No.: 19/708,624

(22) Filed: Jun. 15, 2026

Related U.S. Application Data
(63) Continuation of application No. PCT/US24/60075, filed on Dec. 13, 2024.
(60) Provisional application No. 63/610,507, filed on Dec. 15, 2023.

(51) Int. Cl. C07C 233/56; A61K 31/195; A61K 31/221; A61K 31/395; A61K 31/495; A61K 31/5375; C07D 273/00; C07D 295/185; C07D 498/08

(52) U.S. Cl. CPC C07C 233/56; A61K 31/195; A61K 31/221; A61K 31/395; A61K 31/495; A61K 31/5375; C07D 273/00; C07D 295/185; C07D 498/08

(57) ABSTRACT

This patent document discloses novel oxamates that can act as potent inhibitors of calcium oxalate (CaOx) crystallization and new macrocyclic prodrugs that will offer improved lipophilicity, small size, lower polar surface area, and greatly reduced solvent accessible surface area, all properties known to impart better oral bioavailability. Also disclosed herein are methods of treating diseases associated with abnormal levels of calcium oxalate and its crystallization.

### CROSS REFERENCE TO RELATED APPLICATIONS

This application is a Continuation of International Patent Application No. PCT/US2024/060075, filed on Dec. 13, 2024, which claims priority to U.S. Provisional Patent Application No. 63/610,507, filed on Dec. 15, 2023. Each disclosure is incorporated herein by reference in their entirety.

### TECHNICAL FIELD

The present invention relates to novel compounds that combine molecular mimicry and multisite binding with the capability of inhibiting the crystallization of calcium oxalate (CaOx) in patients with hyperoxaluria. Also disclosed are prodrugs designed to improve their oral bioavailability and methods of preventing kidney stone formation in patients with all types of hyperoxaluria.

### BACKGROUND

Urolithiasis is a common disease worldwide. It occurs when the urine is supersaturated with high levels of calcium, oxalate, phosphate, uric acid, or cystine, leading to the formation of calculi or stones in the urinary system including the kidneys, ureters, and bladder. The incidence of urolithiasis is influenced by a variety of factors including gender, race, metabolic abnormalities, and geographic location. The lifetime risk of urolithiasis is high at about 10 to 15% in the developed world; in some geographic locations like the Middle East, it can be as high as 20 to 25%. In the US, the incidence of urolithiasis has continued to increase over the past 30 years. The most recent assessment of the prevalence of urolithiasis in the US is 8.8%. Among men, the prevalence is higher than women (10.6% vs 7.1%). The risk among white individuals is higher, at approximately three times the risk among black individuals. Stone recurrence is high, estimated at 40% at 5 years and 75% at 20 years. The economic cost associated with kidney stones was estimated to be about $5 billion annually in the US due to hospitalizations, surgery, and lost work time. Urolithiasis has been associated with increased rates of other common diseases and conditions including chronic kidney disease, hypertension, osteoporosis, and obesity.

Calcium oxalate (CaOx) represents the main chemical species of kidney stones, accounting for approximately 70-80% of stones. Because oxalate cannot be metabolized in mammals, it is freely filtered at the glomerulus and secreted by the kidney tubules, eliminated primarily through the urine as an end product of metabolism. The urine that usually contains millimolar concentrations of calcium ions becomes supersaturated with calcium oxalate in hyperoxaluria, resulting in formation of crystals within the tubular lumen. The crystals further aggregate into stones in the urinary tract while some crystals are incorporated into the renal tubule cells and can further migrate into the renal interstitium, which are the clinical hallmarks of the primary hyperoxaluria and can lead to renal failure. Once renal function declines to a certain point, oxalate excretion through the kidneys can be sufficiently compromised that plasma oxalate concentration rises and can rapidly exceed the solubility threshold for calcium oxalate. This leads to systemic deposition of calcium oxalate in tissues other than the kidneys. Such oxalosis can happen in the retina, heart, blood vessel walls, and the brain, and can cause death, if left untreated.

CaOx can be identified in three different crystalline phases: calcium oxalate monohydrate (COM or Whewellite), the most common; calcium oxalate dihydrate (COD or Weddelite), frequency depending on geographic location; and calcium oxalate trihydrate (COT, caoxite), a rare and unstable phase. Disorders that cause increased urinary excretion of oxalate resulting in hyperoxaluria can be classified into primary hyperoxaluria and secondary hyperoxaluria and there are two types of primary hyperoxaluria, type I and type II. Both type I and type II primary hyperoxaluria are rare autosomal recessive disorders where deficiencies in specific enzymes involved in glyoxylate metabolism lead to the overproduction of endogenous oxalate mainly in the liver, and to a small degree in other cells in type II primary hyperoxaluria. Type I primary hyperoxaluria is caused by deficiency in peroxisomal alanine/glyoxylate aminotransferase (AGT) in the liver while type II primary hyperoxaluria is caused by a deficiency in the cytosolic/mitochondrial enzyme glyoxylate/hydroxypyruvate reductase (GR/HPR). A small number of patients with a phenotype similar to that of type I and type II but with normal AGT and GR/HPR enzyme activities have been referred to as non-I, non-II primary hyperoxaluria. The more common secondary forms of hyperoxaluria is caused by too much oxalate or oxalate precursors in the diet or by enhanced intestinal absorption of dietary oxalate due to such underlying disorders as cystic fibrosis, inflammatory bowel diseases, short bowel syndrome, and status post-bariatric surgery. Primary hyperoxaluria is the most severe form and can ultimately lead to end-stage renal failure and death, if not treated.

Management and prevention for calcium oxalate stones are limited. Strategies include general preventive measures, such as increasing fluid intake to increase urine volume, increasing dietary calcium intake, restricting the intake of oxalate-rich foods, using thiazide diuretics and alkalinizing agents that may inhibit growth and aggregation of calcium oxalate. Inhibition of calcium oxalate crystallization has been studied for many years using mostly naturally occurring metal ions, small organic acids, and anionic macromolecules like magnesium, pyruvate, citrate, α-ketoglutarate, phytate. and polymers of aspartic, glutamic, and acrylic acids. Different mechanisms of inhibition have been proposed. For example, magnesium is known to compete with calcium while citrate, pyruvate, α-ketoglutarate, phytate are known as inhibitors of nucleation and crystal growth of calcium oxalate. Citrate is probably the most studied inhibitor and considered to be one of the most potent inhibitors of calcium oxalate crystallization. Clinicians have been advising patients to eat more citrus fruits and juices and prescribing potassium citrate to prevent CaOx stone formation.

There is an ongoing need in the art to develop effective regimen for reducing calcium oxalate crystallization and treating the associated diseases or conditions.

### SUMMARY

The compounds of this patent document address the need. The incorporation of one or more oxamic acids in one single molecule can provide interaction sites/adhesion points to the crystal surface thus providing strong affinity to the crystal surface and prevent the attachment of additional calcium oxalates from binding to the crystal surface hence inhibit crystal growth.

An aspect of this patent document provides a compound or a pharmaceutically acceptable salt thereof, wherein the compound is represented by Formula I,

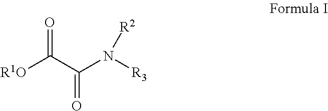

Wherein R¹ is H or C₁₋₈alkyl;
R² is C₁₋₈alkylene-NR^{a}R^{b}, a peptide derivd from one or more natural or unnatural amino acids, or L-[NR^{2′}C(O)COOR^{1′}]m;
L is a linker derived from one or more moieties selected from the group consisting of amino acid(s), C₁₋₈alkylene, S—S, NH—C₁₋₈alkylene, NH—C₁₋₈alkyleneNH, optionally substituted phenyl, optionally substituted 5-10 membered heteroaryl, 5-10 membered carbocyclic, 5-10 membered heterocyclic, wherein L is optionally substituted with one or more moietis selected from the group consisting of COOH, COOC₁₋₆alkyl, CN, halogen, halo-C₁₋₆alkyl, C₁₋₄alkylene-OH, and CN;
m is 1, 2 or 3,
R^{1′} in each instance is independently H or C₁₋₈alkyl;
R^{2′} in each instance is independently H or C₁₋₄alkyl;
R³ is H or C₁₋₈alkyl,
alternatively R² and R³ link up to form a 5-8 membered heterocyclic ring, which is optionally substituted with C₁₋₈alkyl, COOH, COOC₁₋₈alkyl, C(O)NR^{a}R^{b}, or C(O)COOR^{1′}, and
R^{a} and R^{b} are independently H or C₁₋₈alky.

Another aspect provides a compound of Formula II or a pharmaceutically acceptable salt thereof,

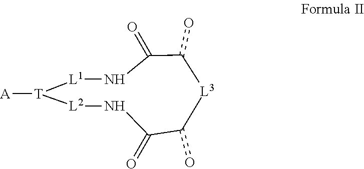

wherein:
T is a bi-functional or tri-functional linker;
A is void when T is a bi-functional linker;
A is

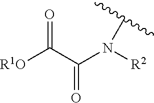

when T is tri-functional linker;
R¹ is H or C₁₋₈alkyl;
R² is H or C₁₋₈alkyl;
L¹ and L² are each a linker;
══O is an optional oxo group,
L³ is a linker.

Another aspect of this patent document discloses a compound of Formula III or a pharmaceutically acceptable salt thereof,

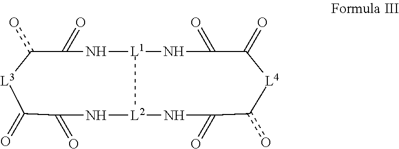

Wherein:
L¹ and L² are each a linker, and are optionally linked with each other;
L³ and L⁴ are each a linker; and
══O is an optional oxo group.

Another aspect of this patent document discloses a pharmaceutical composition comprising the compound described herein or the pharmaceutically acceptable salt, isomer, or prodrug thereof.

Another aspect of this disclosure provides a method of treating a disease in a subject comprising administering to the subject in need a therapeutically effective amount of a compound of Formula I, a pharmaceutically acceptable salt or isomer thereof, or a pharmaceutical composition thereof.

Another aspect provides a method of inhibiting formation of calcium oxalate, comprising administering to a subject in need thereof a therapeutically effective amount of the compound or the pharmaceutically acceptable salt thereof of.

### DESCRIPTIONS OF THE DRAWINGS

FIG. 1 illustrates an assay for measuring inhibition of calcium oxalate crystallization.

FIG. 2 illustrates the synthesis of a compound of Formula I.

FIG. 3 illustrates the synthesis of a compound of Formula III.

### DETAILED DESCRIPTION

Various embodiments of this patent document disclose compounds for inhibition of calcium oxalate crystallization, which is the most common form of kidney stones with all types of hyperoxaluria. Also provided are pharmaceutical compositions of the compounds disclosed herein.

While the following text may reference or exemplify specific embodiments of a compound or a method of treating a disease or condition, it is not intended to limit the scope of the compound or method to such particular reference or examples. Various modifications may be made by those skilled in the art, in view of practical and economic considerations, such as the substitutions of the compound and the amount or administration of the compound for treating or preventing a disease or condition.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “pharmaceutical composition” refers to a mixture of a compound disclosed herein with other chemical components, such as diluents or additional carriers. The pharmaceutical composition facilitates administration of the compound to an organism. Multiple techniques of administering a pharmaceutical composition exist in the art including, but not limited to, oral, injection, aerosol, parenteral, and topical administration. In some embodiments, pharmaceutically acceptable salts of the compounds disclosed herein are provided.

The term “subject” encompasses any animal, but preferably a mammal, e.g., human, non-human primate, a dog, a cat, a horse, a cow, or a rodent. More preferably, the subject is a human.

The term “carrier” refers to a chemical compound that facilitates the incorporation of a compound into cells or tissues.

The term “physiologically acceptable” or “pharmaceutically acceptable” refers to a carrier or diluent that does not abrogate the biological activity and properties of the compound.

The term “therapeutically effective amount” refers to an amount of a compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The term “alkyl” refers to monovalent saturated alkane radical groups particularly having up to about 18 carbon atoms, more particularly as a lower alkyl, from 1 to 8 carbon atoms and still more particularly, from 1 to 6 carbon atoms. The hydrocarbon chain may be either straight-chained or branched. The term “C₁₋₁₀ alkyl” or “C1-C10 alkyl” refers to alkyl groups having 1, 2, 3, 4, 5, 6, 7, 8, 9 or 10 carbon atoms. Similarly, the term “C₁₋₄alkyl” refers to alkyl groups having 1, 2, 3, or 4 carbon atoms. Non-limiting examples of alkyls include groups such as methyl, ethyl, n-propyl, isopropyl, n-butyl, iso-butyl, tert-butyl, n-hexyl, n-octyl, tert-octyl and the like.

The term “alkylene” refers to a divalent hydrocarbon which may be either straight-chained or branched. Different from alkyl which has only one point of bonding with other groups or atoms, alkylene has two points of bonding. Non-limiting examples include groups such as CH₂, (CH₂)₂, CH₂CH(CH₃), and the like. A C₁₋₈alkylene has 1, 2, 3, 4, 5, 6, 7 or 8 carbons. A C₁₋₄alkylene has 1, 2, 3 or 4 carbons.

The term “alkenlene” refers to a divalent double bond-containing hydrocarbon which may be either straight-chained or branched. Non-limiting examples include groups such as CH₂, (CH₂)₂, CHCH, CH₂CHCHCH₂, CH₂CH₂CHCHCH₂CH₂ and the like. A C₂₋₈alkenlene has 2, 3, 4, 5, 6, 7 or 8 carbons. A C₂₋₄ alkylene has 2, 3 or 4 carbons.

The term “carbocycle” or “cycloalkyl” refers to 3 to 10 membered cyclic hydrocarbyl groups having only carbon atoms as ring atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, and multiple ring structures such as adamantanyl, and the like.

The term “heterocycle” refers to 3 to 10 membered substituted or nonsubstituted non-armoatic cyclic groups where one or more carbon ring atoms are replaced with hetero atoms or groups containing heteroatoms (e.g. NH, NC1-C4alkyl O, and S). Nonlimnting examples include pyrrolidine, piperidine, N-methyl-piperizine, and morpholine. Optional substituents include C₁₋₆alkyl, C₁₋₄alkoxy, halogen, haloalkyl, sulfonamido, and amido.

The term “aryl” is intended to mean any stable monocyclic or bicyclic carbon ring of up to 7 members in each ring, wherein at least one ring is aromatic and all ring atoms of the aromatic ring are carbon atoms. Typical aryl groups include, but are not limited to, groups derived from aceanthrylene, acephenanthrylene, anthracene, azulene, benzene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, and the like. Particularly, an aryl group comprises from 6 to 10 or 6 to 14 carbon atoms.

The term “heteroaryl” refers to groups having 5 to 14 ring atoms, preferably 5, 6, 9, or 10 ring atoms, having 6, 10, or 14 π electrons shared in a cyclic array, wherein at least one ring atom contributing to the shared π electrons in the cyclic array is a heteroatom. Typical heteroaryl groups include, but are not limited to, groups derived from acridine, carbazole, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, phenanthridine, phenanthroline, phenazine, phthalazine, phthalimide, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is between 5-15 membered heteroaryl, with 5-10 membered heteroaryl being particularly preferred.

The term “oxo” refers to ═O as a substituent. None-limiting examples of compounds having an oxo group include pyrrolidinone (oxo on pyrrolidine) and cyclopentanone.

The term “treating” or “treatment” of any disease or condition refers, in some embodiments, to ameliorating the disease or disorder (i.e., arresting or reducing the development of the disease or at least one of the clinical symptoms thereof). In some embodiments “treating” or “treatment” refers to ameliorating at least one physical parameter, which may not be discernible by the subject. In some embodiments, “treating” or “treatment” refers to modulating the disease or disorder, either physically, (e.g., stabilization of a discernible symptom), physiologically, (e.g., stabilization of a physical parameter), or both. In some embodiments, “treating” or “treatment” refers to delaying the onset of the disease or disorder, or even preventing the same. “Prophylactic treatment” is to be construed as any mode of treatment that is used to prevent progression of the disease or is used for precautionary purpose for persons at risk of developing the condition.

The term “pharmaceutically acceptable salts” means salts of compounds of the present invention which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, triethanolamine, tromethamine, and N-methylglucamine. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

Most small molecule drugs bind enzymes or receptors in tight and well-defined pockets. On the other hand, designing molecules to inhibit crystal growth is very different. An enzyme or receptor only has a finite number of, often just one, well-defined binding site(s) on it, while on the crystal growth front, be it a ledge or a surface, there are arrays of the same packed grown molecule (e.g., the oxalate molecule on COM). Each such packed grown molecule on a crystal ledge or surface can be replaced by a mimic and could serve as one binding site for the mimic. Many such repeating units form arrays of active/binding sites, thus, allowing for the design of crystallization inhibitors that utilize multisite binding for the enhancement of binding avidity. Multiple binding units linked together in an optimized inhibitor can take advantage of the additive enthalpy of multiple binding site interactions as well as the favorable entropy change of multisite binding events (i.e., enhanced avidity of multivalency).

An aspect of this patent document provides a compound or a pharmaceutically acceptable salt thereof, wherein the compound is represented by Formula I,

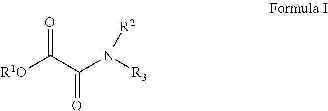

### Wherein:

R¹ is H or C₁₋₈alkyl;
R² is C₁₋₈alkylene-NR^{a}R^{b}, an amide or peptide derived from one or more natural or unnatural amines or amino acids (N terminus of the peptide forms the amide with the oxalate), or L-[N R^{2′}C(O)COOR^{1′}]ₘ;
L is a linker, wherein depending on the value of m, L is connected to one, two or three NHC(O)COOR^{1′} moieties,
m is 1, 2 or 3,
R^{1′} in each instance is independently H or C₁₋₈alkyl;
R^{2′} in each instance is independently H or C₁₋₈alkyl;
R³ is H or C₁₋₈alkyl,
alternatively R² and R³ link up to form a 5-8 membered heterocyclic ring, which is optionally substituted with C₁₋₈alkyl or C(O)COOR^{1′}, and
R^{a} and R^{b} are independently H or C₁₋₈alky.

Linker L may be linear, branched, cyclic, or any combination thereof and may contain or derive from one or more structural components selected from, for example, C₁₋₈alkylene, C₂₋₈alkenlene, NH—C₁₋₈alkylene, NH—C₁₋₈alkylene-NH, O, C(O), NHNH, C(O)NH—C₁₋₈alkylene-NHC(O), 5-8 membered heterocyclic, —(CH₂)ₐC(O)NR^{a}(CH₂)_{b}—, —(CH₂)ₐO(CH₂CH₂O)_{c}—, —(CH₂)ₐheterocyclyl-, —(CH₂)ₐC(O)—, —(CH₂)NR^{a}—, —CR^{a}═N—NR^{a}—, —CR^{a}═N—O—, —CR^{a}═N—NR^{b}—CO—, —N═N—CO—, —S—S—, amino acid, wherein a, b, and c are each an integer selected from 0 to 25, all subunits included; and R^{a} and R^{b} in each instance independently represent hydrogen or a C1-C10 alkyl. Two adjacent components of L can be bonded to each other in the form of an amide, an ester, an ether, a C—C bond, or any chemically feasible connection. It should be noted that the line between L and [NR^{2′}C(O)COOR^{1′}]ₘ does not necessarily indicate a single bond because when L in a branched shape may be connected to two or three [NR^{2′}C(O)COOR^{1′}] moieties. When m is 2 or 3, L can have multiple arms, each connecting to a [NR^{2′}C(O)COOR^{1′}] moiety. The line merely indicates that L as a whole is connected to one or multiple [NR^{2′}C(O)COOR^{1′}] moieties. L (e.g. C₁₋₈alkylene, NH—C₁₋₈alkylene, NH—C₁₋₈alkyleneNH, 3-8 membered carbocycle, 3-8 membered heterycycle, phenyl, 5-10 membered heteroaryl) is optionally substituted with one or more moietis selected from the group consisting of COOH, COOC₁₋₆alkyl, CN, halogen, halo-C₁₋₆alkyl (e.g. CF2, CF3), C₁₋₄akylene-OH, and CN.

In some embodiments, L comprises a moiety derived from an amino acid where the carboxylic acid moiety is reduced into a hydroxyl. L may also be derived from two or more amino acids, where at least one of the carboxylic acids of one of the amino acids is reduced to OH. When an amino acid (e.g. aspartic acid or glutamic acid) has two carboxylic acids, one of the carboxylic acid groups can be reduced to OH in R². Nonlimiting examples of components of L derived from amino acides include lysinol and cystinol.

In some embodiments, L together with the nitrogen it is attached to forms a heterocyclic ring. For example, L can be piperazinyl moiety where the two nitrogens are part of an amide (e.g. terminal oxylamides).

In some embodiments, the linker L is or contains a component selected from NHNH, C₂₋₆alkylene, C₂₋₆alkenlene, C(O)NH—C₀₋₆alkylene-NHC(O), and C(O)-piperazinyl-C(O).

In some embodiments, R² is L-NR^{2′}C(O)COOR^{1′}. In some embodiments, L is C₂₋₆alkylene and R³ is H. In some embodiments, R³ is C₁₋₄alkyl (e.g. methyl, ethyl, propyl). In some embodiments, any R^{2′} is independently H or C₁₋₄alkyl (e.g. methyl, ethyl, propyl). In some embodiments, all R^{2′} in formula I are C₁₋₄alkyl (e.g. methyl, ethyl, propyl). In some embodments, m is 1. In some embodments, m is 2. In some embodiments, R¹ is C₁₋₄alkyl (e.g. methyl, ethyl, propyl). In some embodiments, any carboxylate groups in Formula I can be in the form of an ester C₁₋₄alkyl (e.g. methyl, ethyl, propyl). In some embodiments, all carboxylate groups in Formula I are in the form of an ester C₁₋₄alkyl.

In some embodiments, R² and R³ link up to form an optionally substituted heterocyclic ring. In some embodiments, the heterocyclic ring is morpholine or piperazine, wherein the ring is optionally substituted with H, C₁₋₈alkyl or C(O)COOR^{1′}. R^{1′} in each instance is independently H or C₁₋₈alkyl.

In some embodiments, R² is C₁₋₈alkylene-NR^{a}R^{b}. In some embodiments, R² is C₂₋₆alkylene-NH₂.

In some embodiments, R² is a peptide derived from one or more amino acids. For example, R² can be derived from an amino acid where the carboxylic acid moiety is reduced into a hydroxyl. R² may also be derived from two or more amino acids, where at least one of the carboxylic acids of one of the amino acids is reduced to OH. When an amino acid (e.g. aspartic acid or glutamic acid) has two carboxlic acids, one of the carboxlic acid groups can be reduced to OH in R². The N-terminus of the peptide forms the amide group with the oxalate. When R² is a peptide derived from one or more amino acids, the C termins of the peptide can be in the form of an amide (e.g. C(O) NR^{a}R^{b}, R^{a} and R^{b} are independently H or C₁₋₈alky or an ester (e.g. C(O)C₁₋₈alky). The amino acid in each instance can be a natural or unnatural one. In some embodiments, the amino acids are selected from one or more of histidine, isoleucine, leucine, lysine, methionine, phenylalanine, threonine, tryptophan, valine, alanine, asparagine, aspartic acid, glutamic acid, serine, arginine, cysteine, cystine, glutamine, glycine, proline, tyrosine, β-amino-propionic acid, and γ-amino-butyric acid. Two adjacent amino acids may be linked by any chemically feasible means including for example an amide or a disulfide.

In some embodiments, R² is L-NHC(O)COOR^{1′} and comprises a moiety derived from one or more amino acids, the scope of which is as described above. Nonlimting examples include lysinol, cystinol and other amino acides with the carboxylic acid reduced into a hydroxyl group.

In some embodiments, R¹ and R³ are each independently C₁₋₄alkyl. In some embodiments, R² is L-[NR^{2′}C(O)COOR^{1′}]ₘ, wherein R^{1′} and R^{2′} are each independently C₁₋₄alkyl. When R¹, R³, R^{1′} and R^{2′} are C₁₋₄alkyl, they can be same or different. In some embodiments, R¹, R³, R^{1′} and R^{2′} are independently methyl or ethyl. In some embodiments, all amide nitrogens are independently alkylated by C₁₋₄alkyl.

In any embodiments disclosed herein, when the compound contains one or more sterocenter, each stereocenter can be independently R or S in configuration.

In some embodiments, L is branched and is connected to two NHC(O)COOR^{1′}. L may thus comprises a trifunctional or multifunctional linkage moiety derived from for example, lysine, 1,3-diamino-2-propanol, spermidine, spermine, or therospermine. In some embodiments, L comprises one or two lysines for extending the linker and connect to one, two, or more NHC(O)COOR^{1′}. Of course, the linker may contain one or more additional components such as C₁₋₈alkylene, NH—C₁₋₈alkylene, and NH—C₁₋₈alkylene-NH as described above.

Nonlimiting examples of R² include the following:

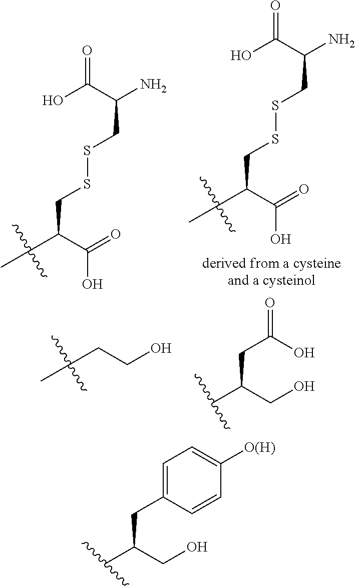

Nonlimiting examples of NR²R³ include the following:

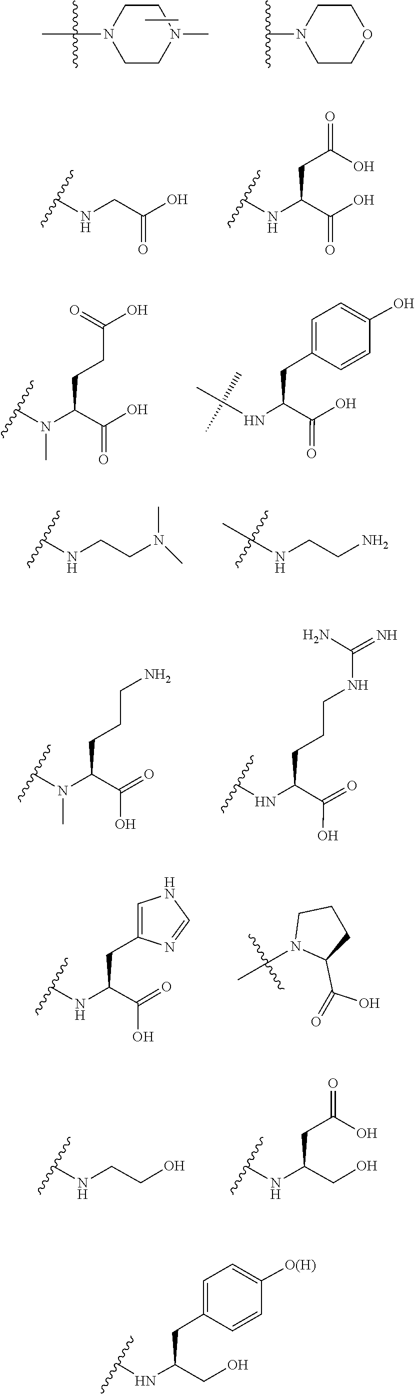

Nonlimiting examples of L include the following:

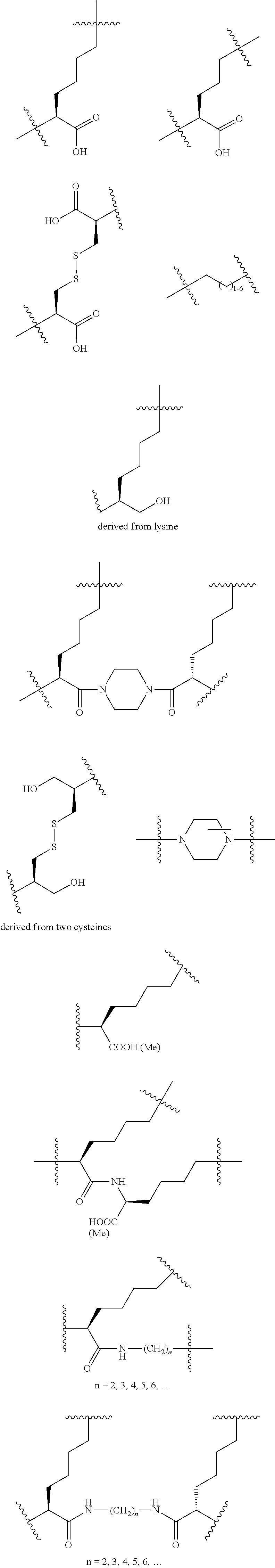

In some embodiments, L is branched, m is 3 and L is connected to three NHC(O)COOR^{1′}. For instance, as shown in the formula below, L includes interconnected L¹ and L², which further link to three additional oxamate moieties besides the original first one (shown in the box). L¹ and L² each may contain one or more components as described above for linkers. In some embodiments, one or both of L¹ and L² contain a moiety derived from lysine.

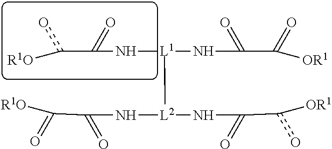

In some embodiments, L¹ and L² are connected via a linear component such as C₁₋₈alkylene, NH—C₁₋₈alkylene, and NH—C₁₋₈alkylene-NH. In some embodiments, L¹ and L² are connected via a component containing a ring such as a 5-6 membered cyclic ring or a 5-6 membered heterocyclic ring. In some embodiments, the connection between L¹ and L² is or contain a component selected from NHNH, C₂₋₆alkylene, C₂₋₆alkenlene, C(O)NH—C₀₋₆alkylene-NHC(O), and C(O)-piperazinyl-C(O).

A related aspect provides a cyclized form of the compound of Formula I. In some embodiments, the cyclized compound is represented by Formula II.

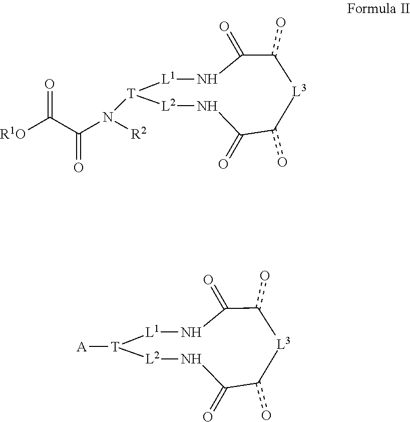

Wherein:
T is a bi-functional or tri-functional linker;
A is void when T is a bi-functional linker;
A is

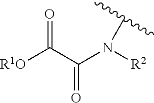

when T is tri-functional linker;
R¹ is H or C₁₋₈alkyl;
R² is H or C₁₋₈alkyl;
══O is an optional oxo group,

In the cyclic structures where the carbonyl of an oxamate moiety is in the form of an ester, the ester linkage can be enzymatically cleaved under in vivo condition. Further as illustrated below, when the enzymatic cleavage gives rise to a glycoamide moiety, the α-carbon of the glycoamide can be further oxidized under in vivo condition into a carbonyl, resulting in a newly formed oxamate moiety.

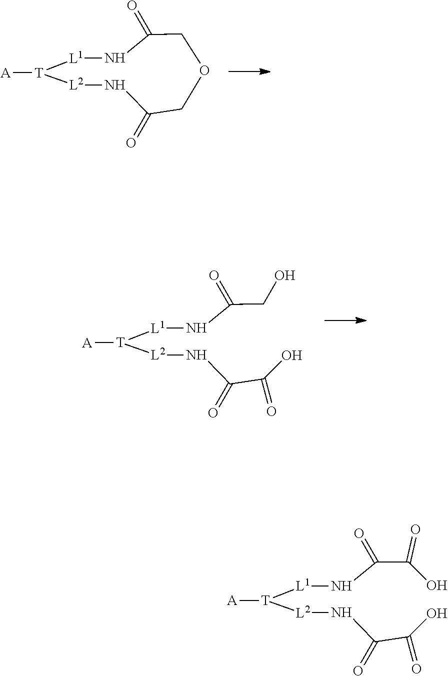

L¹, L² and L³ may be linear, branched, cyclic, or any combination thereof and may contain or derive from one or more structural components selected from, for example, C₁₋₈alkylene, C₂₋₈ alkenlene, NH—C₁₋₈alkylene, NH—C₁₋₈alkylene-NH, O, NHNH, C(O)NH—C₁₋₈alkylene-NHC(O), 5-8 membered heterocyclic, —(CH₂)ₐC(O)NR(CH₂)_{b}—, —(CH₂)ₐO(CH₂CH₂O)_{c}—, —(CH₂)ₐheterocyclyl-, —(CH₂)ₐC(O)—, —(CH₂)ₐNR^{a}—, —CR^{a}═N—NR^{a}—, —CR^{a}═N—O—, —CR^{a}═N—NR^{b}—CO—, —N═N—CO—, —S—S—, amino acid(s), wherein a, b, and c are each an integer selected from 0 to 25, all subunits included; and R^{a} and R^{b} in each instance independently represent hydrogen or a C1-C10 alkyl.

In some embodiments, the compound is in the form of Formula II-a. T is part of L¹ or L².

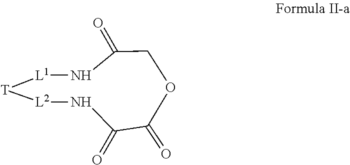

In some embodiments of Formula II, the oxo ══O is absent, and L³ is O or NR^{a}R^{b}, wherein R^{a} and R^{b} are independently H or C₁₋₈alkyl.

In some embodiments, the compound is in the form of Formula II-b, wherein T is trifunctional linker.

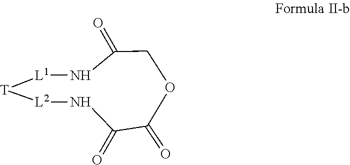

In some embodiments of Formula II, the oxo group is present, L³ is O—C₁₋₈alkylene-O. T by itself or together with components of one or both of L¹ and L² may be derived by any suitable branched structures including for example lysine, 1,3-diamino-2-propanol, spermidine, and spermine.

Another related aspect provides a cyclic structure related to the above described compounds. The cyclic structure is represented by Formula III.

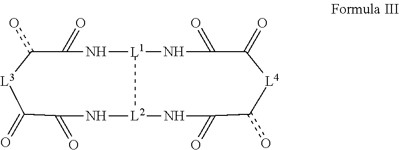

wherein:
L¹ and L² are each a linker, and are optionally linked with each other;
L³ and L⁴ are each a linker; and
══O is an optional oxo group.

L¹, L², L³, and L⁴ are each a linker, which may be linear, branched, cyclic, or any combination thereof and may contain or derive from one or more structural components selected from, for example, C₁₋₈alkylene, C₂₋₈alkenlene, NH—C₁₋₈alkylene, NH—C₁₋₈alkylene-NH, O, NHNH, C(O)NH—C₁₋₈alkylene-NHC(O), 5-8 membered heterocyclic, —(CH₂)ₐC(O)NR^{a}(CH₂)_{b}—, —(CH₂)ₐO(CH₂CH₂O)—, —(CH₂)ₐheterocyclyl-, —(CH₂)ₐC(O)—, —(CH₂)ₐNR^{a}—, —CR^{a}—NR^{a}—, —CR^{a}═N—O—, —CR═N—NR^{b}—CO—, —N═N—CO—, —S—S—, amino acid, wherein a, b, and c are each an integer selected from 0 to 25, all subunits included; and R^{a} and R^{b} in each instance independently represent hydrogen or a C1-C10 alkyl.

Preferably, L³ and/orL⁴ are linked to the oxamate carbonyl via an ester linkage. L³ and/or L⁴ can also be linked to the carbon having an optional oxo via an oxygen, so that it can be enzymatically cleaved or oxidized into a carbonyl group as explained above.

In some embodiments, the two oxo groups are present, L³ and L⁴ independently each comprises O—C₁₋₈alkylene-O. In some embodiments, L¹ and L² are independently derived from or comprise one or more moieties selected from the group consisting of amino acid(s), C₁₋₈alkylene, NH—C₁₋₈alkylene, NH—C₁₋₈alkyleneNH, optionally substituted phenyl, optionally substituted 5-10 membered heteroaryl, 5-10 membered carbocyclic, 5-10 membered heterocyclic.

In some embodiments, L¹ and L² are linked with each other. In some embodiments, the connection between L¹ and L² is or contain one or more components selected from NHNH, C₂₋₁₂alkylene, C₂₋₁₂alkenlene, C(O)NH—C₀₋₁₂alkylene-NHC(O), and C(O)-piperazinyl-C(O).

In some embodiments of Formula III, the oxo groups are absent, and L³ and L⁴ are each O. L¹ and L² are linked with each other. The scope of possible linkage between L¹ and L² are the same as described in the compounds where two oxo groups are present.

Another aspect provides a compound or a pharmaceutically acceptable salt thereof, wherein the compound is represented by Formula IV,

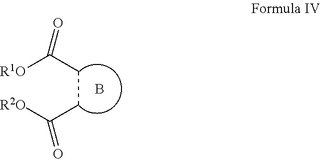

Wherein:
R¹ and R² are each independently H or C₁₋₈alkyl;
B is an optionally substituted ring, and
the dotted line indicates an optional bond.

Ring B can be a 3-8 membered carbocyle, a 3-8 membered heterocycle, an 6-10 membered aryl, or a 5-10 membered heteroaryl. In some embodiments, B is a 5 membered heteroaryl. In some embodiments, B is an optionally substituted imidazole. In some embodiments, the ring is a 3-8 membered carbocyle, a 3-8 membered heterocycle. In some embodiments, the dotted line is present as part of an aromatic aryl or heteraryl.

Nonlimiting substituens of the ring include C₁₋₆alkyl, OC₁₋₄alkyl, NR^{a}R^{b}, halo C₁₋₄alkyl, halogen (F, Cl, Br or I), OH, and CN. some embodiments, B is substituted C₁₋₄alkyl.

Nonlimiting examples of Formula IV include the following, which can be in acid, ester, or other salt forms.

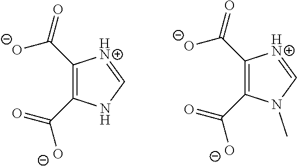

Another aspect provides a pharmaceutical composition comprising a therapeutically effective amount of the compound or the pharmaceutically acceptable salt thereof described herein.

The following structures illustrate an anion form of the compounds of Formula I. Of course, the compounds may also be in other forms including for example an acid, zwitterionic form, an ester (e.g. C₁₋₆ alkyl ester), a salt or a neutral form.

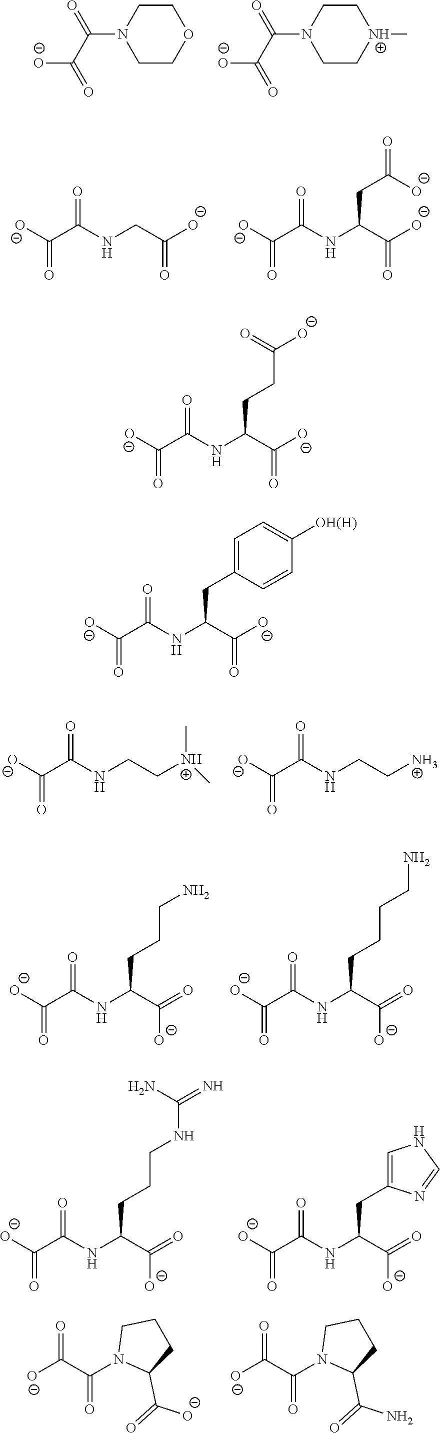

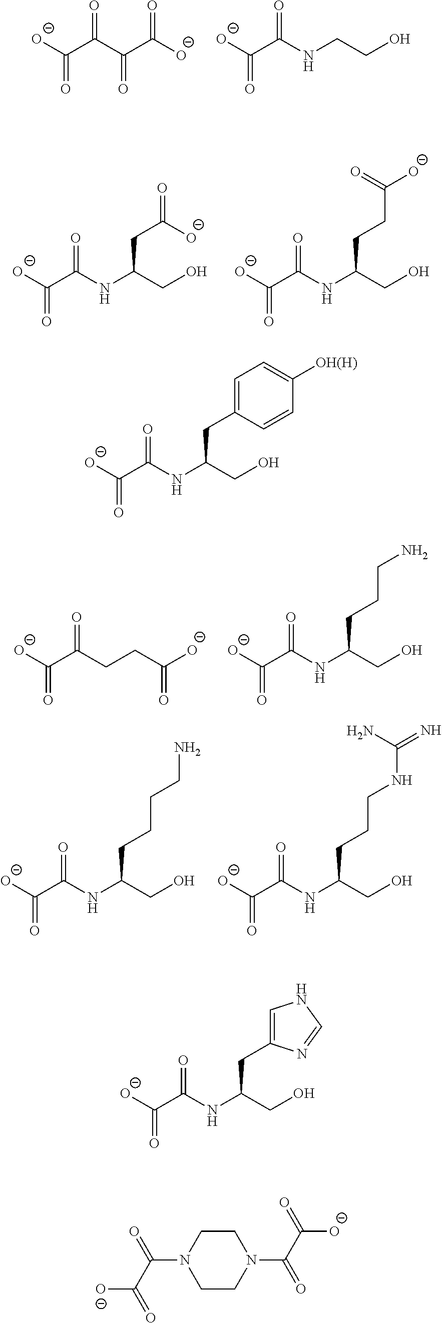

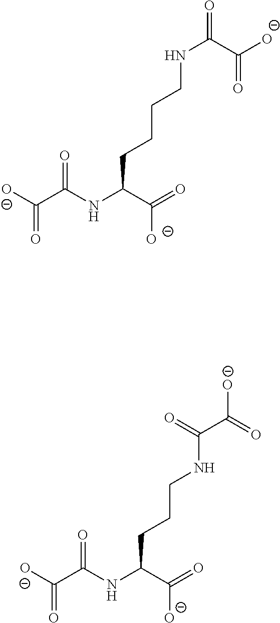 [SMILES: O=C([O-])C(=O)NCCCC[C@H](NC(=O)C(=O)[O-])C(=O)[O-] ; O=C([O-])C(=O)NCCC[C@H](NC(=O)C(=O)[O-])C(=O)[O-]]

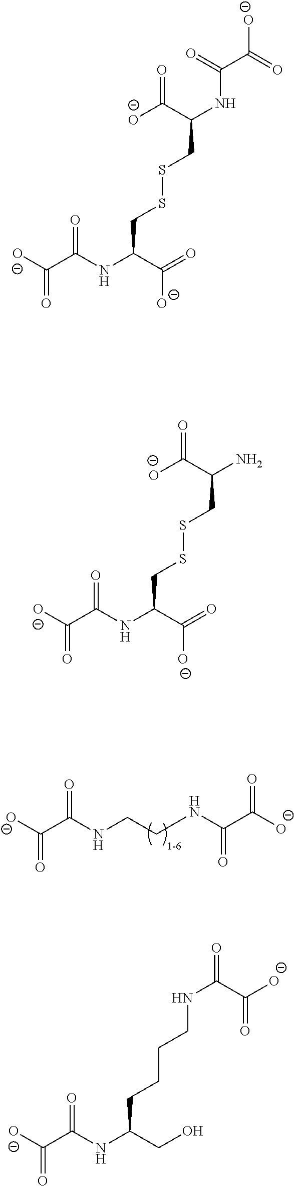

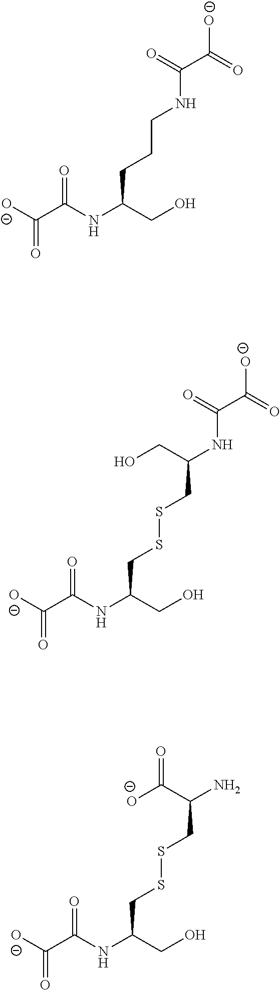 [SMILES: O=C([O-])C(=O)NCCC[C@@H](CO)NC(=O)C(=O)[O-] ; O=C([O-])C(=O)N[C@H](CO)CSSC[C@@H](CO)NC(=O)C(=O)[O-] ; N[C@@H](CSSC[C@@H](CO)NC(=O)C(=O)[O-])C(=O)[O-]]

The following structures illustrate cyclic forms of compounds disclosed herein.

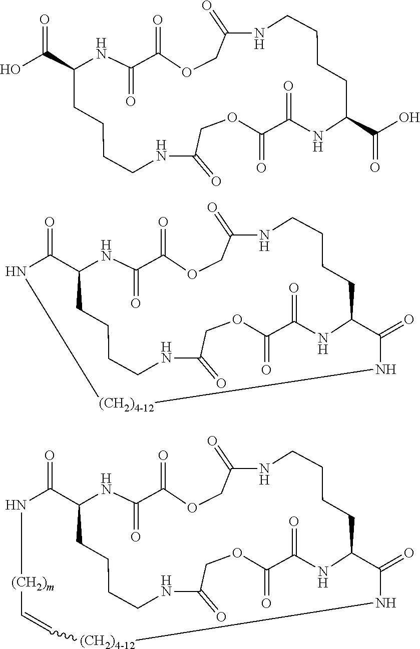

Nonlimiting examples of compounds of Formula I, II, or III include the following. The nitrogen of one or more amides in R² can be alkylated (e.g. methylated or ethylated). The subscript m and n are each independently 2, 3, 4, 5, 6, 7, 8, 9, 10, 11, 12, 13, 14 or 15.

TABLE 1
(1-20—Formula I; 21-26—Formula II; 27-28—Formula III)

| Com- | | | |
|---|---|---|---|
| pound | R1 | R2/R3 | L |
| 1 | H | CH₂CH₃NH₂/H or Methyl | |
| 2 | CH₂CH₃ | CH₂CH₃NH₂/H or Methyl | |
| 3 | H | 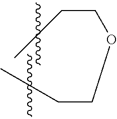 [SMILES: *CCOCC*] | |
| 4 | CH₂CH₃ | 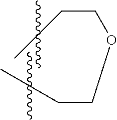 [SMILES: *CCOCC*] | |
| 5 | H | 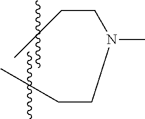 [SMILES: *CCN(C)CC*] | |
| 6 | CH₂CH₃ | 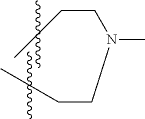 [SMILES: *CCN(C)CC*] | |
| 7 | H | —CH(R)CO₂H | |
| 8 | CH₂CH₃ | —CH(R)CO₂H | |
| 9 | H | Lys(Oxalyl)-OH/H or Methyl | |
| 10 | CH₂CH₃ | Lys(COCO₂Et)-OMe/H or Methyl | |
| 11 | H | (CH₂)ₙ—NHCOCO₂H/H | n = |
| | | | 2,3,4,5,6, |
| 12 | CH₂CH₃ | (CH₂)ₙ—NHCOCO₂H/H | n = |
| | | | 2,3,4,5,6, |
| 13 | H | Lys(COCO₂H)-Lys | |
| | | (COCO₂H)—OH/H or Methyl | |
| 14 | CH₂CH₃ | Lys(COCO₂Et)-Lys | |
| | | (COCO₂Et)—OMe/H orMethyl | |
| 15 | H | Lys(COCO₂H)—(CH₂)n- | n = |
| | | NHCOCO₂H/H orMethyl | 2,3,4,5,6, |
| | | | . . . |
| 16 | CH₂CH₃ | Lys(COCO₂Et)—(CH₂)n- | n = |
| | | NHCOCO₂Et/H orMethyl | 2,3,4,5,6, |
| | | | . . . |
| 17 | H | Lys(COCO₂H)-Piperazine- | |
| | | ψ[NCO]-Lys(COCO₂H)— | |
| | | COCO₂H/H or Methyl | |
| 18 | CH₂CH₃ | Lys(COCO₂Et)-Piperazine-ψ[NCO]- | |
| | | Lys(COCO₂Et)—COCO₂Et/ | |
| | | H or Methyl | |
| 19 | H | Lys(COCO₂H)—(CH₂)ₙ—ψ[NHCO]- | n = |
| | | Lys(COCO₂H)—COCO₂H/H | 2,3,4,5,6, |
| | | or Methyl | |
| 20 | CH₂CH₃ | Lys(COCO₂H)—(CH₂)n-ψ[NHCO]- | n = |
| | | Lys(COCO₂H)—COCO₂H/H | 2,3,4,5,6, |
| | | or Methyl | ... |
| 21 | | Cyclo[24][[OCH₂CO-Lys | |
| | | (COCO₂—)—OMe]2] | |
| 22 | | Cyclo[2ₘ + 26[[OCOCO-Lys | |
| | | (COCO₂—(CR′R″)ₘ—)—OMe]2] | |
| 23 | | Cyclo[2n + 14][OCH₂CO— | |
| | | NH(CH₂)ₙNHCOCO₂—)]₂] | |
| 24 | | Cyclo[2m + 2n + 16[[OCOCO— | |
| | | NH(CH₂),NH(COCO₂— | |
| | | (CR′R″)ₘ—)]₂] | |
| 25 | CH₂CH₃ | Cyclo[n+14][Lys(OCCH₂—)— | n = |
| | | (CH₂)ₙ—NHOCCO₂-]/H or Methyl | 2,3,4,5,6, |
| | | | . . . |
| 26 | CH₂CH₃ | Cyclo[n + m + 15][Lys | n = |
| | | (OCCO₂—)—(CH₂)ₙ— | 2,3,4,5,6, |
| | | NHOCCO₂— | m = |
| | | (CR′R″)ₘ—]/H or Methyl | 1,2,3,4,5, |
| | | | R, R′ = H, |
| | | | or C₁₋₆ |
| | | | alkyl |
| | | | (e.g. CH₃, |
| | | | ethyl ) |
| 27 | | Bicyclo[11,11,n + 4][Lys | n = |
| | | (OCCH₂—)—(CH₂)ₙ— | 2,3,4,5,6, |
| | | NHOCCO₂—]/H or Methyl | . . . |
| 28 | | Bicyclo[m + 12, m + 12, n + 4] | n = |
| | | [[Lys(OCCO₂—)— | 2,3,4,5,6, |
| | | (CH₂)ₙ—NHOCCO₂— | m = 1,2,3,4,5, |
| | | (CR′R″)ₘ—]/H or Methyl | R, R″ = H, |
| | | | or C₁-₆ |
| | | | alkyl (e.g. CH₃, |
| | | | ethyl) |

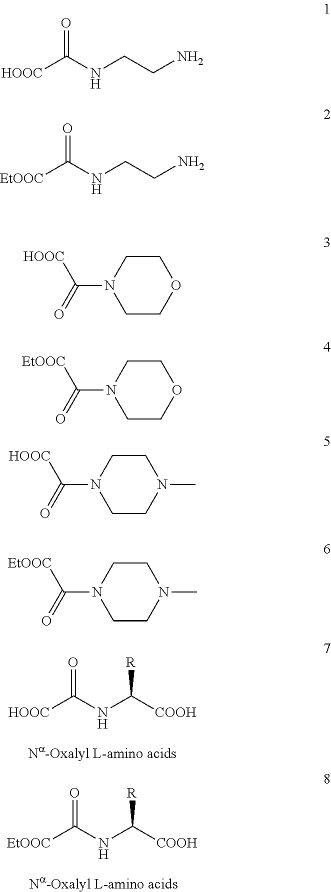

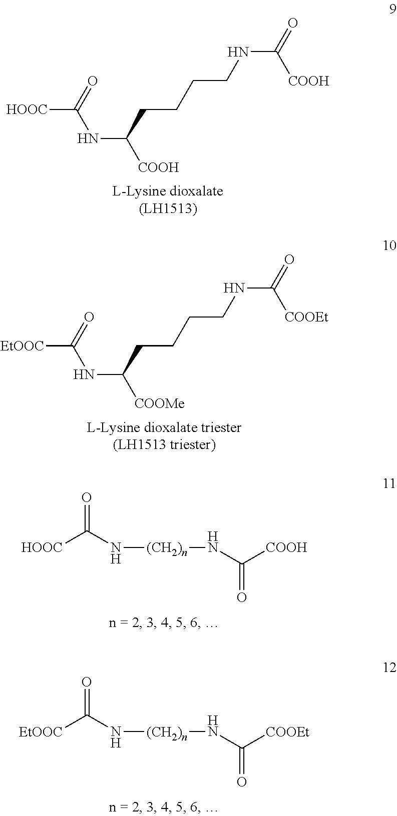

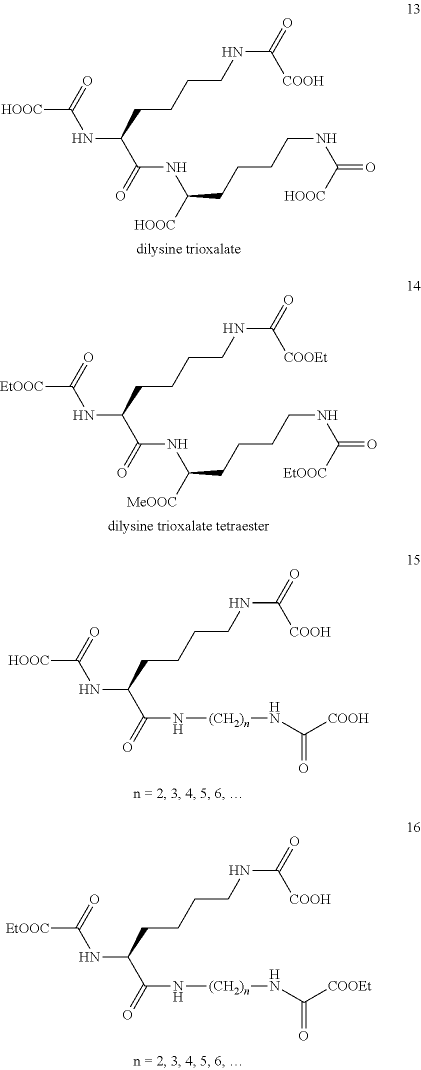

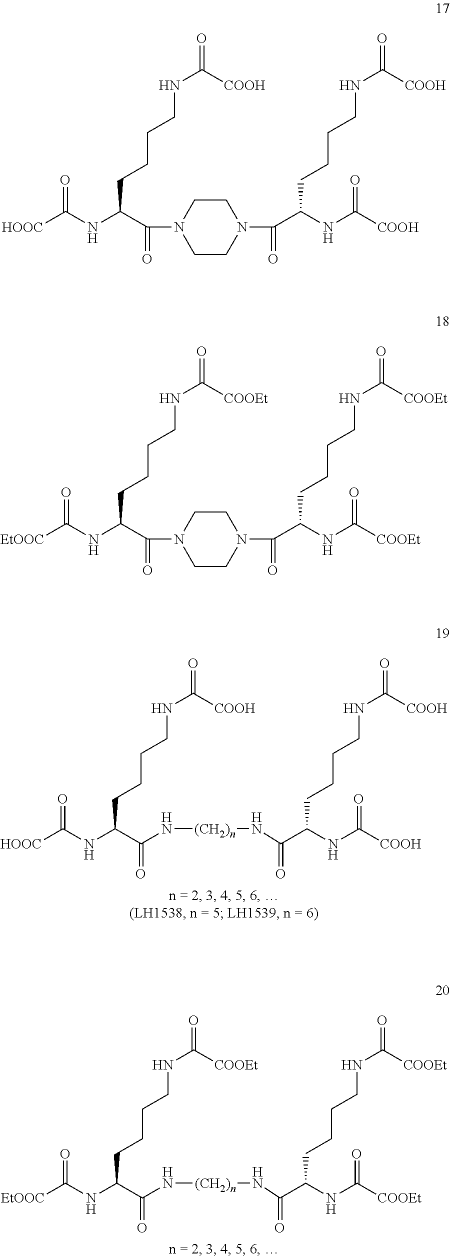

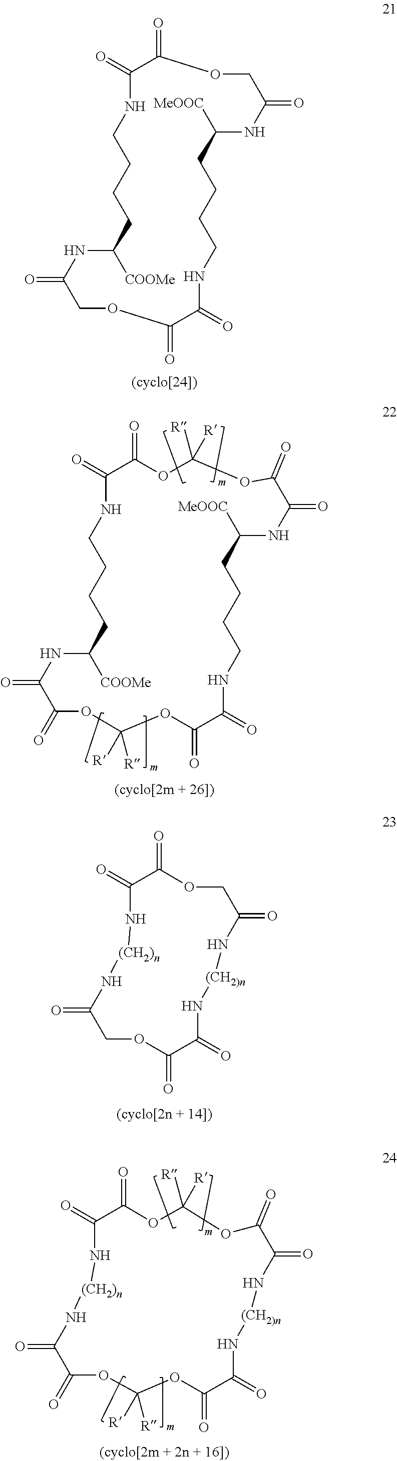

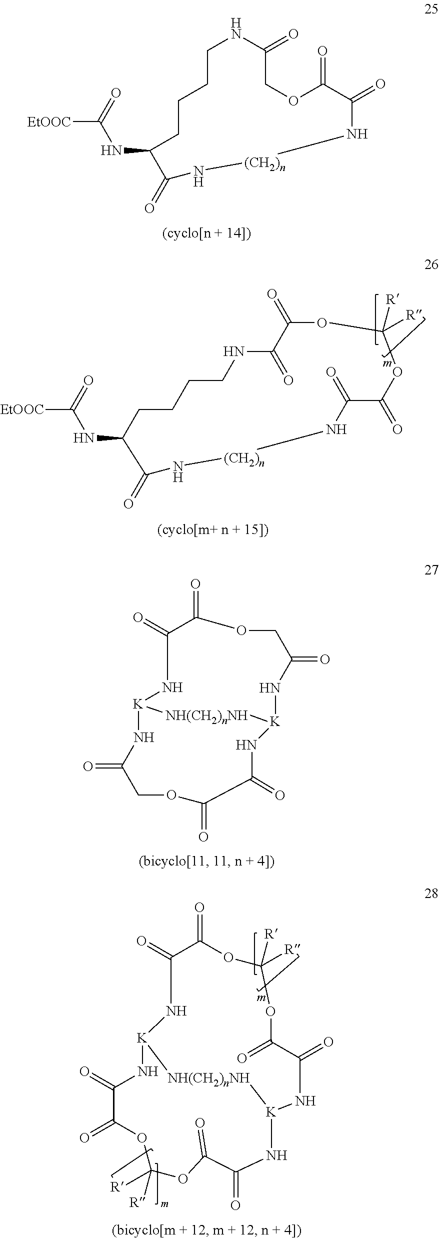

Nonlimiting examples of N-methyl analogs include the following:

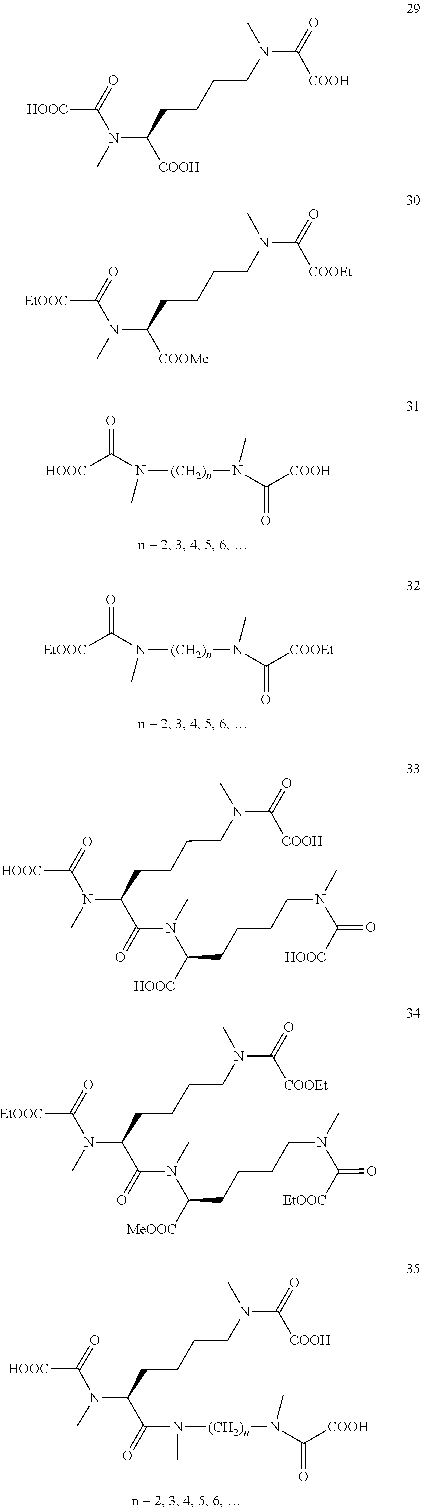

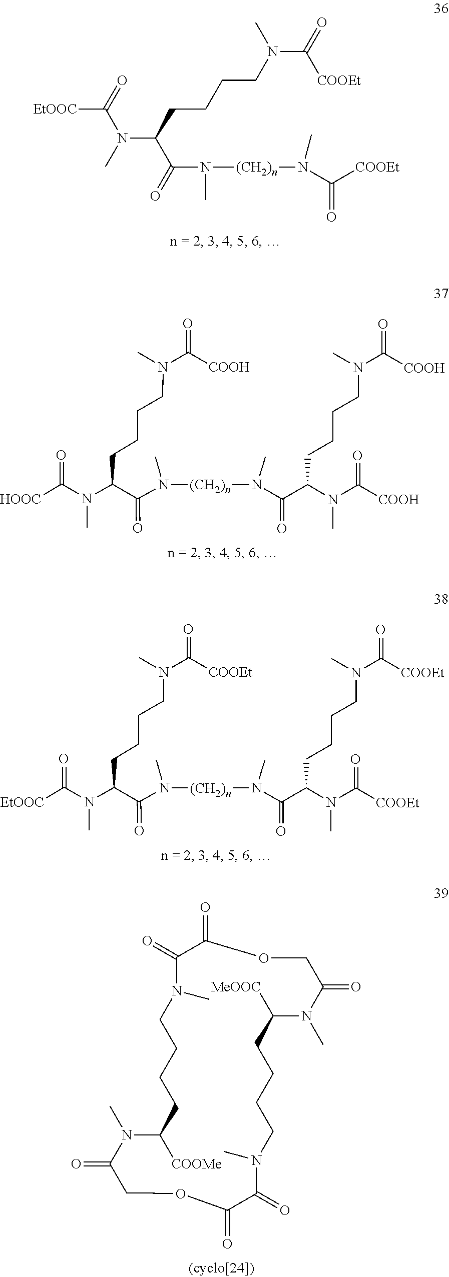

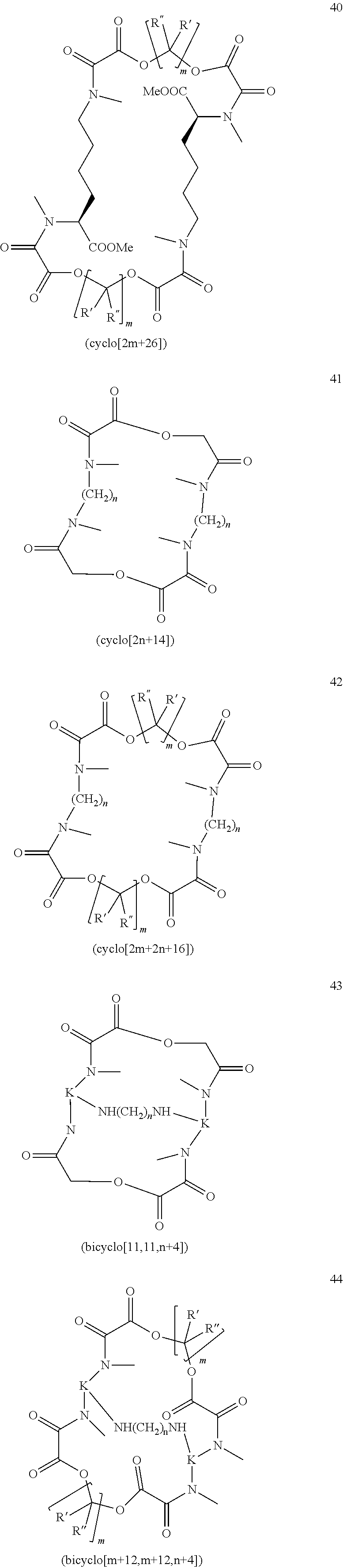

Another aspect of the present disclosure provides a pharmaceutical composition containing a therapeutically effective amount of the above-described compound and a pharmaceutically acceptable carrier.

The pharmaceutical composition may also contain one or more physiologically acceptable surface-active agents, additional carriers, diluents, excipients, smoothing agents, suspension agents, film forming substances, and coating assistants, or a combination thereof; and a composition disclosed herein. Acceptable additional carriers or diluents for therapeutic use are well known in the pharmaceutical art, and are described, for example, in Remington's Pharmaceutical Sciences, 18th Ed., Mack Publishing Co., Easton, PA (1990), which is incorporated herein by reference in its entirety. Preservatives, stabilizers, dyes, sweeteners, fragrances, flavoring agents, and the like may be provided in the pharmaceutical composition. For example, sodium benzoate, ascorbic acid, and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, microcrystalline cellulose, crystallized cellulose, mannitol, light anhydrous silicate, magnesium aluminate, magnesium metasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

The pharmaceutical compounds described herein can be administered to a human patient per se, or in pharmaceutical compositions where they are mixed with other active ingredient(s), as in combination therapy, or suitable carriers or excipient(s). In some embodiments, a dosage form includes those forms in which the compound is administered per se. In addition, a dosage form may include a pharmaceutical composition. In any case, the dosage form may comprise a sufficient amount of the compound to treat a disease as part of a particular administration protocol, as would be understood by those of skill in the art. Techniques for formulation and administration of the compounds of the instant application may be found in “Remington's Pharmaceutical Sciences,” Mack Publishing Co., Easton, PA, 18th edition, 1990.

The pharmaceutical compositions may be manufactured in a manner that is itself known, e.g., by means of conventional mixing, dissolving, granulating, dragee-making, levigating, emulsifying, encapsulating, entrapping or tabletting processes.

Pharmaceutical compositions may be formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, diluents, carriers, and excipients may be used as suitable and as understood in the art; e.g., in Remington's Pharmaceutical Sciences, above.

Another aspect of this disclosure provides a method of inhibiting formation of calcium oxalate, comprising administering to a subject in need thereof a therapeutically effective amount of the compound or the pharmaceutically acceptable salt thereof. The compound or the pharmaceutically acceptable salt thereof can be administered after the diagnosis of a relevant disease or condition. Alternatively, the compound can be administered prophylactically, especially for those who are at risk of having a higher than normal level of calcium oxalate, or at risk of developing a disease or condition associated with a higher than normal level of calcium oxalate. A related aspect provides a method of treating a disease selected from the group consisting of urolithiasis, kidney disease, hypertension, osteoporosis, obesity. The method includes comprising administering to a subject in need thereof a therapeutically effective amount of the compound or the pharmaceutically acceptable salt thereof of disclosed herein. In some embodiments, the method is directed to inhibition of calcium oxalate formation in retina, heart, blood vessel walls, and/or brain of the subject.

The compound disclosed herein or a pharmaceutical composition thereof may also be used in combination with or include one or more other therapeutic agents, for example selected from citrate, pyruvate, α-ketoglutarate, phytate.

Pharmaceutical compositions suitable for administration include compositions where the active ingredients are contained in an amount effective to achieve its intended purpose. In some embodiments, a therapeutically effective amount of a compound is an amount effective to treat a viral infection, for example, in a mammalian subject (e.g., a human). The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication, and other factors which those skilled in the medical arts will recognize. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved adverse side effects disappear. The dosage may range broadly, depending upon the desired effects and the therapeutic indication. Typically, dosages may be about 10 microgram/kg to about 100 mg/kg body weight, preferably about 100 microgram/kg to about 10 mg/kg body weight. Alternatively, dosages may be based and calculated upon the surface area of the patient, as understood by those of skill in the art.

The exact formulation, route of administration and dosage for the pharmaceutical compositions can be chosen by the individual physician in view of the patient's condition. (see e.g., Fingl et al. 1975, in “The Pharmacological Basis of Therapeutics”, which is hereby incorporated herein by reference in its entirety, with particular reference to Ch. 1, p. 1). In some embodiments, the dose range of the composition administered to the patient can be from about 0.5 to about 1000 mg/kg of the patient's body weight. The dosage may be a single one or a series of two or more given in the course of one or more days, as is needed by the patient. In instances where human dosages for compounds have been established for at least some conditions, those same dosages, or dosages that are about 0.1% to about 500%, more preferably about 25% to about 250% of the established human dosage may be used. Where no human dosage is established, as will be the case for newly discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. The daily dosage regimen for an adult human patient may be, for example, an oral dose of about 0.1 mg to 2000 mg of the active ingredient, preferably about 1 mg to about 500 mg, e.g. 5 to 200 mg. In other embodiments, an intravenous, subcutaneous, or intramuscular dose of the active ingredient of about 0.01 mg to about 100 mg, preferably about 0.1 mg to about 60 mg, e.g. about 1 to about 40 mg is used. In cases of administration of a pharmaceutically acceptable salt, dosages may be calculated as the free acid. In some embodiments, the composition is administered 1 to 4 times per day. Alternatively, the compositions may be administered by continuous intravenous infusion, preferably at a dose of up to about 1000 mg per day. As will be understood by those of skill in the art, in certain situations it may be necessary to administer the compounds disclosed herein in amounts that exceed, or even far exceed, the above-stated, preferred dosage range to effectively and aggressively treat particularly aggressive diseases or infections. In some embodiments, the compounds will be administered for a period of continuous therapy, for example for a week or more, or for months or years.

Dosage amount and interval may be adjusted individually to provide plasma levels of the active moiety, which are sufficient to maintain the antibiotic effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen, which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered may be dependent on the subject being treated, on the subject's weight, the severity of the infection, the manner of administration and the judgment of the prescribing physician.

Compositions disclosed herein can be evaluated for efficacy and toxicity using known methods. For example, the toxicology of the compound may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound formulated in a compatible pharmaceutical carrier may also be prepared, placed in an appropriate container, and labeled for treatment of an indicated condition.

In some embodiments, in the pharmaceutical industry, it is standard practice to provide substantially pure material when formulating pharmaceutical compositions. Therefore, in some embodiments, “substantially pure” refers to the amount of purity required for formulating pharmaceuticals, which may include, for example, a small amount of other material that will not affect the suitability for pharmaceutical use. In some embodiments, the substantially pure compound contains at least about 96% of the compound by weight, such as at least about 97%, 98%, 99%, or 100% of the compound.

### EXAMPLES

### Example 1

Synthesis of oxamic acid from amines. The following scheme illustrate the synthesis of oxamic acid ethyl ester.

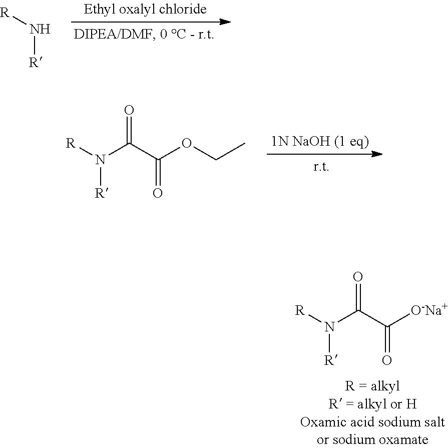

A suspension of an organic amine free base or hydrochloride salt (1 mmol) in DMF (1 mL) containing DIPEA (1.1 eq., additional eq. needed if salts are used) was added drop-wise to an ice-cooled solution of ethyl oxalyl chloride (1.1 mmol, 1.1 eq., additional eq. needed if multiple amines present). The reaction mixture was stirred at r.t. for 3 h. After the reaction was completed as monitored by TLC and/or LCMS, ice water was added to quench the excess the reagent and the product was extracted with dichloromethane three times. The combined organic phase was washed with sat. NaHCO₃ twice, 5% citric acid twice, and dried over anhydrous Na₂SO₄ and purified by ISCO flash chromatography on a prepacked silica gel column, eluted with gradient of MeOH in DCM to give the desired intermediate oxamic acid ethyl ester.

The intermediate ethyl ester obtained above was hydrolyzed with 1 N NaOH (1 eq to each ester present) at room temperature and hydrolysis was usually completed within 2 h and the reaction mixture was lyophilized to afford the desired oxamic acid as its sodium salt.

### Example 2

The following scheme illustrates the synthesis of L-Lysine N^{α},N^{ε}-dioxalate.

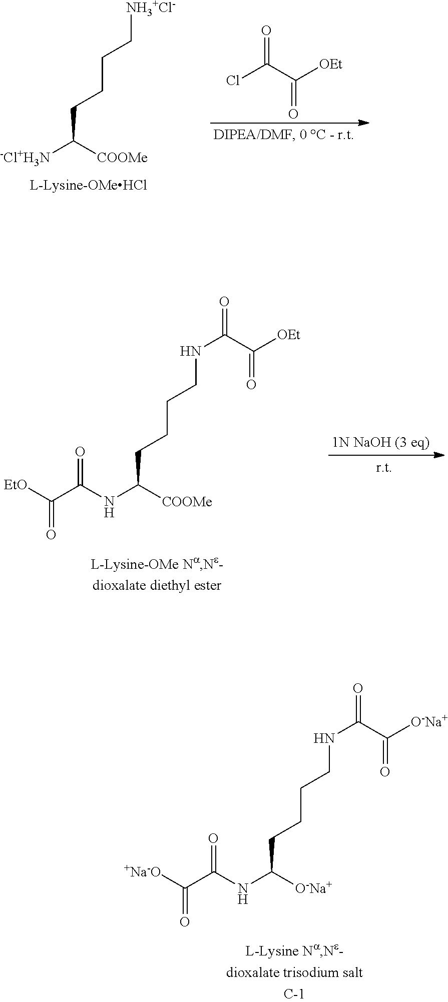

A suspension of L-lysine methyl ester hydrochloride (233 mg, 1 mmol) in DMF (1 mL) containing DIPEA (582 mg, 4.5 mmol, 4.5 eq.) was added drop-wise to an ice-cooled solution of ethyl oxalyl chloride (600 mg, 4.4 mmol, 4.4 eq.). The reaction mixture was stirred at r.t. for 3 h. After the reaction completion as shown by LCMS, ice water (~10 mL) was added to quench the excess the reagent and the product was extracted with dichloromethane (3×10 mL). The combined organic phase was washed with sat. NaHCO₃ twice, 5% citric acid twice, and dried over anhydrous Na2SO4 and purified by ISCO on a 12 g silica gel column, eluted with 0-10% MeOH in DCM to give the desired product N^{α},N^{ε}-bis(ethoxyoxalyl)-L-Lysine methyl ester (DW-I-006A) (230 mg, 64% yield). ¹H NMR (400 MHz, CDCl₃) δ 7.58 (d, J=8.4 Hz, 1H), 7.15 (s, 1H), 4.64 (td, J=8.0, 5.1 Hz, 1H), 4.44-4.26 (m, 4H), 3.80 (s, 3H), 3.35 (q, J=6.8 Hz, 2H), 1.97 (m, 1H), 1.80 (m, 1H), 1.71-1.54 (m, 2H), 1.41 (m, 8H). ¹³C NMR (101 MHz, CDCl₃) δ 171.5, 160.7, 160.0, 156.7, 156.3, 63.4, 63.2, 52.7, 52.3, 39.4, 31.8, 28.5, 22.4, 14.0.

The intermediate esters obtained above (C-1, 60 mg, 0.167 mmol) was hydrolyzed with 1 N NaOH (0.5 mmol, 3 eq) at room temperature and hydrolysis was complete within 2 h and the reaction mixture was lyophilized to afford the desired L-Lysine N^{α},N^{ε}-dioxalate trisodium salt (60 mg, 100% yield, DW-I-007). ¹H NMR (400 MHz, D₂O) δ 4.05 (dd, J=8.2, 4.9 Hz, 1H), 3.11 (t, J=7.1 Hz, 2H), 1.74 (dt, J=13.6, 6.7 Hz, 1H), 1.64 (dq, J=14.7, 7.8 Hz, 1H), 1.45 (tt, J=9.2, 5.0 Hz, 2H), 1.27 (p, J=7.7 Hz, 2H). ¹³C NMR (100 MHz, D₂O) δ 178.1, 166.0, 165.7, 165.2, 164.1, 55.1, 31.8, 28.3, 22.7. HRMS (ESI⁻) m/z calcd. for C₁₀H₁₃N₂O₈ 289.0677, found 289.0670 ([M-H]⁻).

### Example 3

Procedure for the synthesis of 1,n-bis(L-lysinamido)alkane N^{α},N^{α′},N^{ε},N^{ε′}-tetraoxalates. The following scheme illustrates the synthesis of a compound containing four oxalate moieties.

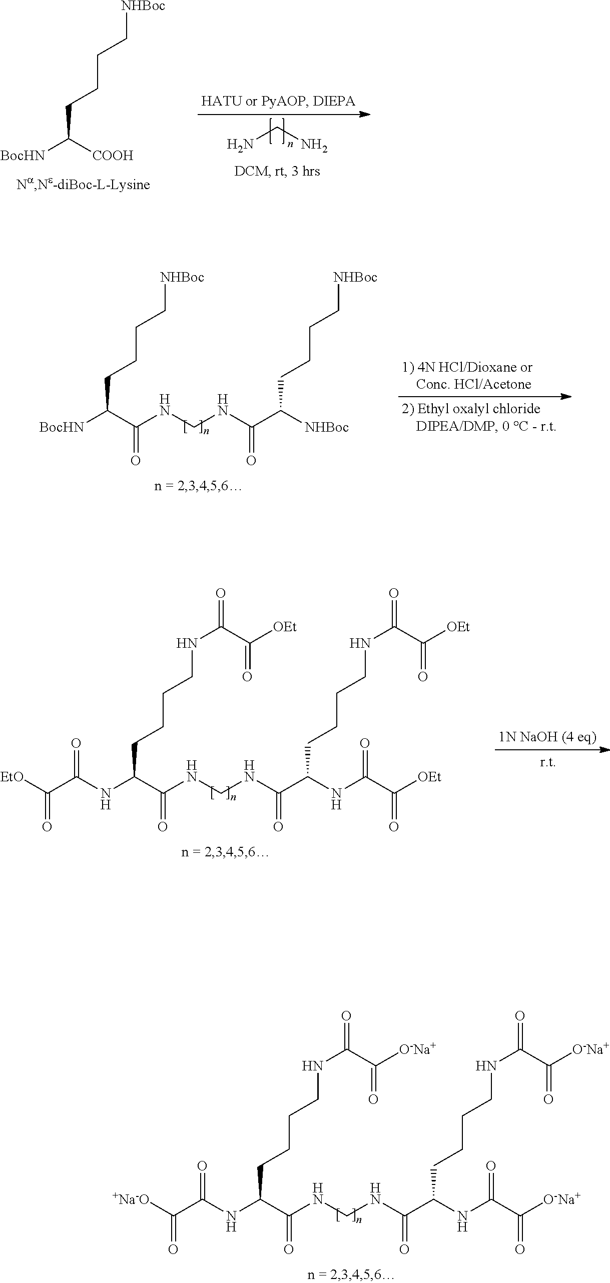

To the suspension of N^{α},N^{ε}-diBoc-L-lysine (2 mmol), HATU (2 mmol)and DIPEA (2 mmol) in DMF at 0° C. was added an organic diamine (1 mmol). Additional equivalents or DIPEA was added if an organic diamine salt was used. The reaction mixture was stirred at r.t. and monitored using TLC and/or LCMS. After reaction was completed (~2 h), the reaction mixture was quenched with cold water. After decanting the liquid phase, the sticky residue was purified by ISCO flash chromatography on a prepacked silica gel column, eluted with gradient of 0-40% ethyl acetate in DCM to afford the desired Boc protected 1,n-bis(L-lysinamido)alkane intermediate.

Boc protected 1,n-bis(L-lysinamido)alkane intermediate obtained above was deprotected using 4 N HCl in dioxane or concentrated HCl in acetone to afford the 1,n-bis(L-lysinamido)alkane intermediate containing 4 primary amines as their HCl salt. Following the general procedure for the synthesis of oxamic acid from amines, the 1,n-bis(L-lysinamido)alkane intermediate was acylated with ethyl oxalyl chloride and the resulting tetraethyl ester was hydrolyzed with four equivalents of NaOH to obtain the desired 1,n-bis(L-lysinamido)alkane N^{α}, N^{α′},N^{ε},N^{ε′}-tetraoxalates as the sodium salts after lyophilization.

### Example 4

The following scheme illustrates the synthesis of N,N′-(1,5-pentylene)bis(L-Lysinamide) N^{α}, N^{α′},N^{ε},N^{ε′}-tetraoxalate (C-2)

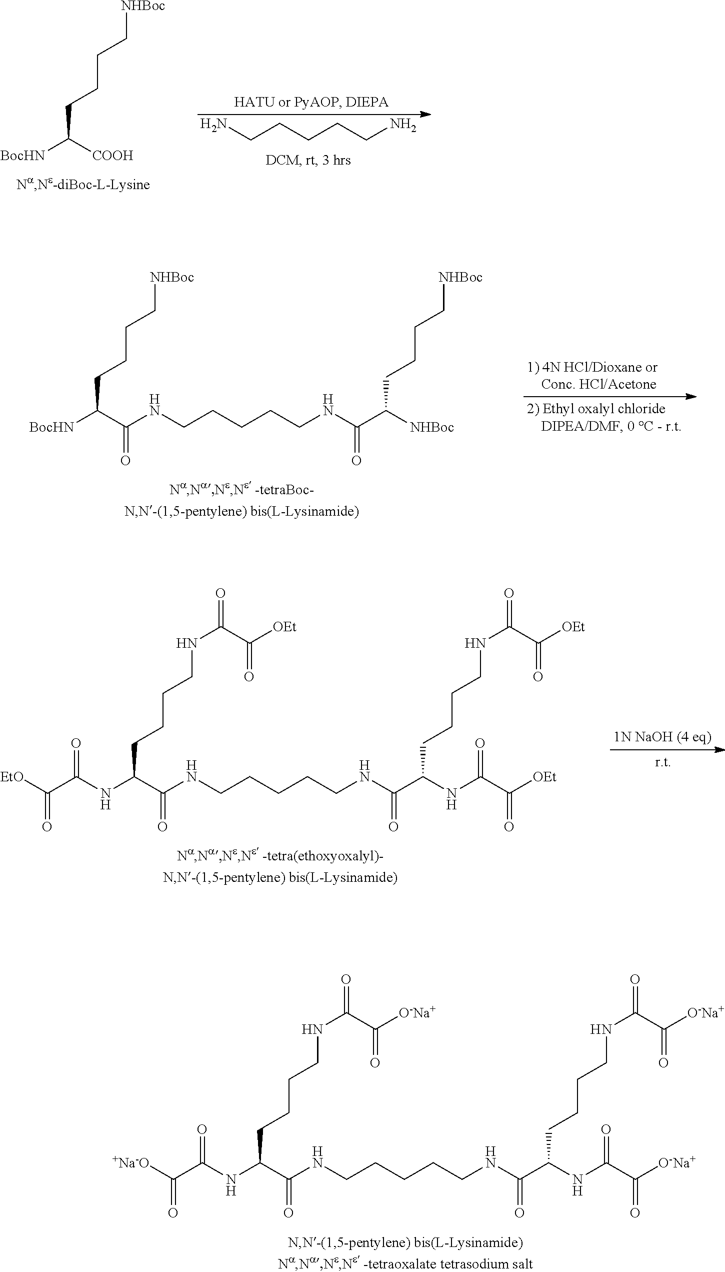

To the suspension of N^{α},N^{ε}-diBoc-L-Lysine (692 mg, 2 mmol), 1,5-pentylenediamine hydrochloride (175 mg, 1 mmol) and DIPEA (646 mg, 5 mmol) in DMF (6 mL) at 0° C. was added HATU (955 mg, 2.5 mmol). The reaction mixture was stirred at r.t. and monitored using LCMS. After completion of the reaction (2 h), the reaction was quenched with cold water (20 mL). After decanting the liquid phase, the sticky residue was purified by ISCO (40 g column) with 15 min gradient of 0-40% ethyl acetate in DCM at 40 mL/min to afford the Boc protected N,N′-(1,5-pentylene)bis(L-Lysinamide) intermediate.

The tetraBoc protected N,N′-(1,5-pentylene)bis(L-Lysinamide) intermediate obtained above (1 mmol) was dissolved in DCM (10 mL) and cooled to 0° C., to which was then added dropwise 4 N HCl in dioxane (5.4 mL, 5 eq). After stirring at r.t. for 2 h, the reaction mixture was concentrated to removed reagent and solvent to afford the N,N′-(1,5-pentylene)bis(L-Lysinamide) HCl salt.

The N,N′-(1,5-pentylene)bis(L-Lysinamide) HCl salt (1 mmol) was dissolved in DMF (6 mL) with the addition of 10 eq of DIPEA. The solution was cooled to 0° C. and then ethyl oxalyl chloride (6 eq.) in THF (3 mL) was added dripwise. The reaction mixture was stirred at 0° C. for 1 h and then at r.t. for 2 h. LCMS monitoring indicated the presence of triacylated intermediate, suggesting incomplete reaction. An additional 3 eq. of ethyl oxalyl chloride and 5 eq. of DIPEA were added at 0° C. The reaction mixture was stirred at 0° C. for 30 min and then at r.t. for 1 h until completion of the reaction as monitored by LCMS. The reaction was quenched with the addition of cold water (20 mL) and then extracted with DMC (3×20 mL). The combined organic phase was washed with 5% citric acid, saturated NaHCO₃, and water before dried over anhydrous Na₂SO₄. Removal of organic solvent gave the crude product, which was purified using ISCO on a 40 g prepacked silica gel column with 15 min gradient of 0-10% Methanol in DCM at 40 mImin to afford the N^{α},N^{α′},N^{ε},N^{ε′}-tetra(ethoxyoxalyl)-N,N′-(1,5-pentylene)bis(L-Lysinamide) intermediate as a light yellow oil (630 mg, 83% yield over 3 steps).

¹H NMR (400 MHz, CDCl₃) δ 7.90 (d, J=8.3 Hz, 2H), 7.45 (t, J=6.2 Hz, 2H), 7.05 (t, J=5.7 Hz, 2H), 4.48 (q, J=7.3 Hz, 2H), 4.34 (q, J=7.1 Hz, 8H), 3.39-3.33 (m, 4H), 3.33-3.15 (m, 4H), 1.94 (dq, J=14.5, 7.3 Hz, 2H), 1.85-1.71 (m, 4H), 1.63 (q, J=7.3 Hz, 4H), 1.57-1.34 (m, 20H), 1.34-1.21 (m, 4H). ¹³C NMR (101 MHz, CDCl₃) δ 171.2, 170.6, 160.6, 156.8, 63.3, 63.2, 60.4, 53.5, 53.4, 50.7, 39.5, 39.2, 31.9, 28.6, 28.5, 23.6, 22.6, 14.2, 14.0, 14.0.

To the solution of N^{α},N^{α′},N^{ε},N^{ε′}-tetra(ethoxyoxalyl)-N,N′-(1,5-pentylene)bis(L-Lysinamide) intermediate (320 mg) in methanol (4 mL) as added 1 N NaOH (1.69 mL, 4 eq) at 0° C. The reaction mixture was stirred at 0° C. for 30 min and at r.t. for 2 h or until starting material disappears as monitored by LCMS. The reaction mixture was concentrated and the residue was dissolved in water (5 mL) before lyophilization to give the desired product N,N′-(1,5-pentylene)bis(L-Lysinamide) N^{α}, N^{α′},N^{ε},N^{ε′}-tetraoxalate tetrasodium salt as an off-white solid (270 mg, 87% yield).

¹H NMR (400 MHz, D₂O) δ 4.12 (dd, J=8.3, 5.9 Hz, 2H), 3.16-3.02 (m, 8H), 1.83-1.64 (m, 4H), 1.46 (q, J=7.2 Hz, 4H), 1.39 (q, J=7.4 Hz, 4H), 1.35-1.23 (m, 4H), 1.20 (d, J=7.3 Hz, 2H).

### Example 5

The following scheme illustrates the synthesis of N,N′-(1,6-hexylene)bis(L-Lysinamide) N^{α}, N^{α′},N^{ε},N^{ε′}-tetraoxalate

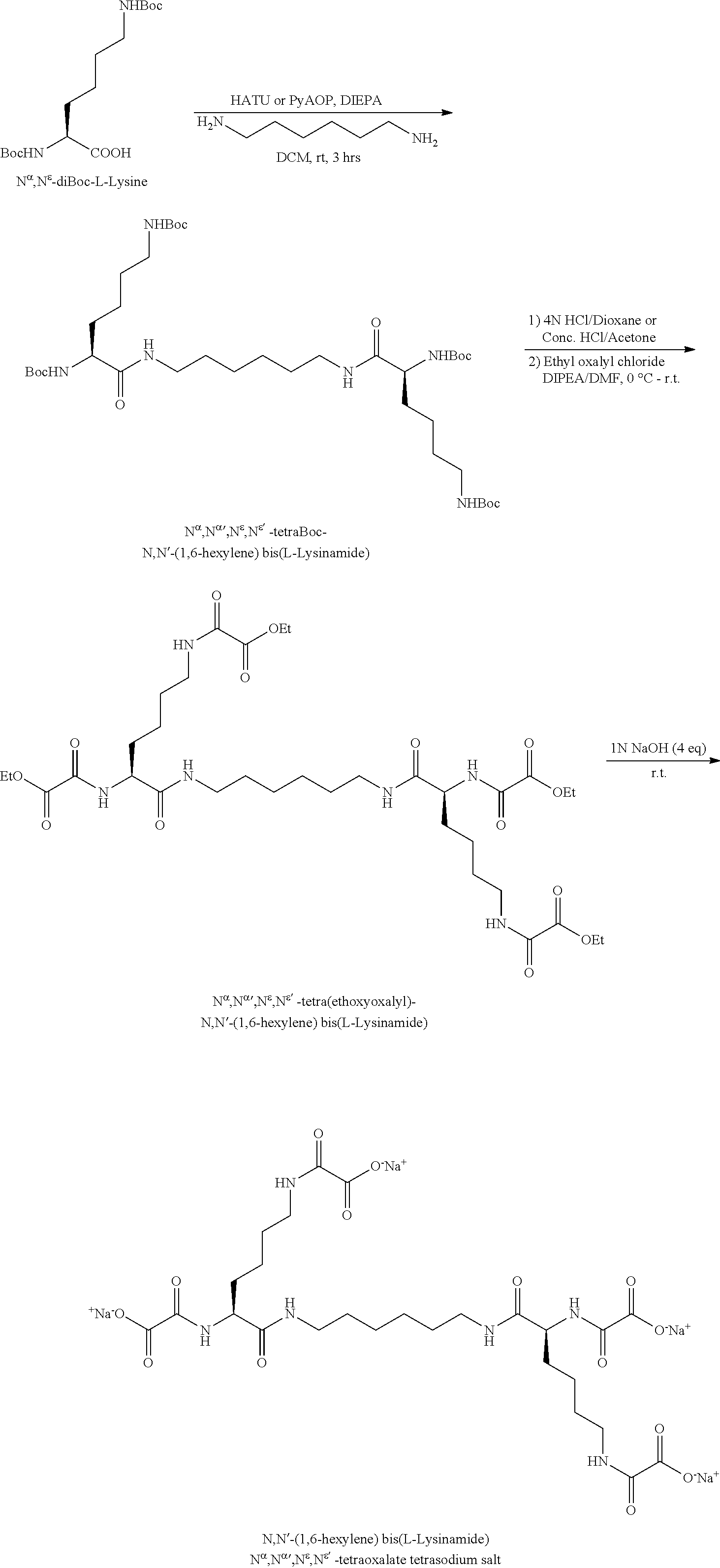

To the suspension of N^{α},N^{ε}-diBoc-L-Lysine (692 mg, 2 mmol), 1,6-hexylenediamine (116 mg, 1 mmol) and DIPEA (388 mg, 3 mmol) in DMF (3 mL) at 0° C. was added HATU (955 mg, 2.5 mmol). The reaction mixture was stirred at r.t. and monitored using LCMS. A second aliquot of HATU (300 mg) and DIPEA (200 mg) were added at r.t. after 1 h. After completion of reaction (total 2 h), the reaction was quenched with cold water (20 mL). After decanting the liquid phase, the sticky residue was purified by ISCO (40 g column) with 15 min gradient of 0-100% ethyl acetate in hexane at 40 mL/min to afford the Boc protected N,N′-(1,6-hexylene)bis(L-Lysinamide) intermediate (671 mg, 43% yield).

The tetraBoc protected N,N′-(1,6-hexylene)bis(L-Lysinamide) intermediate obtained above (620 mg, 0.8 mmol) was dissolved in DCM (5 mL) and cooled to 0° C., to which was then added dropwise 4 N HCl in dioxane (4.8 mL, 6 eq). After stirring at r.t. for 3 h, the reaction mixture was concentrated to removed reagent and solvent to afford the N,N′-(1,6-hexylene)bis(L-Lysinamide) HCl salt (410 mg, 79% yield).

The N,N′-(1,6-hexylene)bis(L-Lysinamide) HCl salt (410 mg, 0.79 mmol) was dissolved in DMF (5 mL) with the addition of 10 eq of DIPEA. The solution was cooled to 0° C. and then ethyl oxalyl chloride (5 eq.) in THF (5 mL) was added dripwise. The reaction mixture was stirred at 0° C. for 30 min and then at r.t. for 2 h until completion of the reaction as monitored by LCMS. The reaction was quenched with the addition of cold water (20 mL) and then extracted with DMC (3×20 mL). The combined organic phase was washed with 5% citric acid, saturated NaHCO₃, and water before dried over anhydrous Na₂SO₄. Removal of organic solvent gave the crude product, which was purified using ISCO on a 24 g prepacked silica gel column with 15 min gradient of 0-10% Methanol in DCM to afford the N^{α},N^{α′},N^{ε},N^{ε′}-tetra(ethoxyoxalyl)-N,N′-(1,6-hexylene)bis(L-Lysinamide) intermediate as a light yellow oil (360 mg, 59% yield).

To the solution of N^{α},N^{α′},N^{ε},N^{ε′}-tetra(ethoxyoxalyl)-N,N′-(1,6-hexylene)bis(L-Lysinamide) intermediate (290 mg, 0.375 mmol) in methanol (4 mL) as added 1 N NaOH (1.5 mL, 4 eq) at 0° C. The reaction mixture was stirred at 0° C. for 30 min and at r.t. for 4 h or until starting material disappears as monitored by LCMS. The reaction mixture was concentrated and the residue was dissolved in water (5 mL) before lyophilization to give the desired product N,N′-(1,6-hexylene)bis(L-Lysinamide) N^{α}, N^{α′},N^{ε},N^{ε′}-tetraoxalate tetrasodium salt as an off-white solid (168 mg, 60% yield).

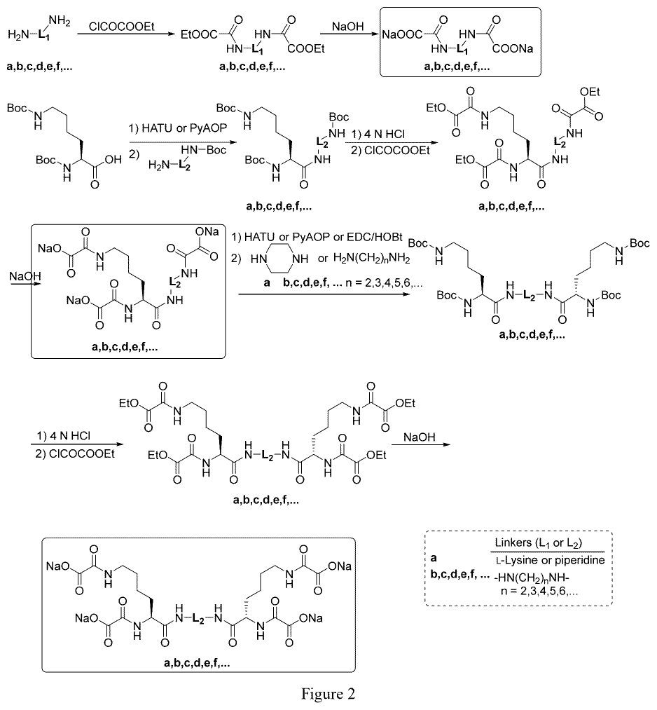

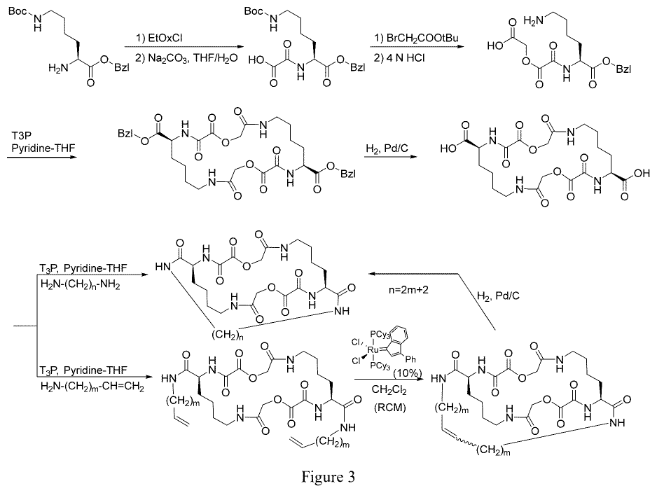

FIG. 2 also illustrates the synthesis of a compound of Formula I. FIG. 3 illustrates the synthesis of a compound of Formula III in a cyclic form.

### Example 6

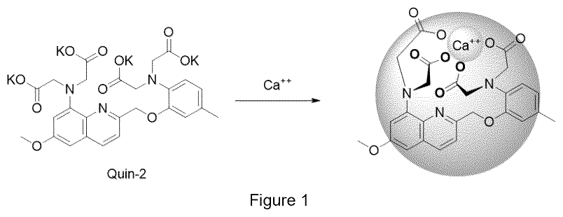

Calcium oxalate (CaOx) crystallization inhibition assays in HEPES buffer, pH 6.8 include Assay A and Assay B. Assay A: Fluorescence-based assay using Quin-2 to measure the concentration of free calcium in solution (Ex 340 nm/Em 495 nm) (FIG. 1). An example assay protocol is shown below.

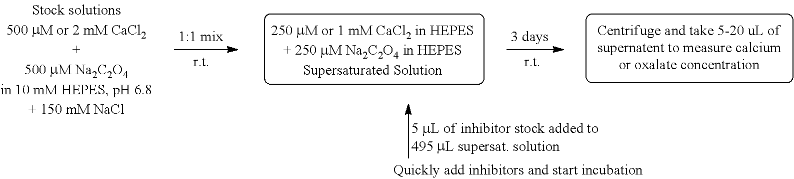

Assay B: Spectrophotometric assay using EnzyChrom™ oxalate assay kit from Bioassay Systems (EOXA-100) to measure the concentration of free oxalate in solution at OD 595 nm.

For CaOx crystallization inhibition assay, equal volumes of 2 mM of CaCl₂) was combined with 500 mM of Na₂Ox to generate 250 mM supersaturated solutions of CaOx in 10 mM HEPES buffer, pH 6.8 containing 150 mM NaCl. The higher concentration of Ca⁺⁺ utilized in this assay was designed to mimic the typical calcium levels found in human urine. Following incubation with varying concentrations of inhibitors at r.t. for 72 h and subsequent centrifugation, the EnzyChrom™ oxalate assay kit (EOXA-100, BioAssay Systems) was employed to measure spectrophotometrically the remaining oxalate concentration in solution at 595 nm. This assay determined the EC₅₀ for each inhibitor. This assay was used to determine the activity of about 50 oxamate derivatives synthesized. Separately, dose response curves can be obtained for calcium concentrations and inhibitors concentrations with supersaturated solution: 250 μM CaCl₂+250 μM Na2C2O4 in 10 mM HEPES, pH 6.8+150 mM NaCl; Calcium concentration in supernatent after centrifuge was measured using Quin2 via a fluorescence assay (Ex 340 nm/Em 495 nm).

Table 1 lists the EC₅₀'s obtained using this assay for various oxamate derivatives, along with other structure features such as linker length and number of OMA units for SAR analysis.

TABLE 2
Activity of oxamate derivatives as
inhibitors of CaOx crystallization

| | Linker | # | EC₅₀ | | |
|---|---|---|---|---|---|
| Compd # | length | OMA | (μM)^{a} | Ratio^{b} | pEC₅₀^{c} |
| Citrate | — | — | 665 | 1 | 3.18 |
| 1 | — | 1 | 377^{d} | 2 | 3.42 |
| 9 | 5 | 2 | 63.8 | 10 | 4.20 |
| 11b | 2 | 2 | 127^{d} | 5 | 3.89 |
| 11e | 5 | 2 | 145 | 5 | 3.84 |
| 11f | 6 | 2 | 135 | 5 | 3.87 |
| 17 | 5/2/5 | 4 | 25.7 | 26 | 4.59 |
| 19b | 5/2/5 | 4 | 58.7 | 11 | 4.23 |
| 19c | 5/3/5 | 4 | 3.43 | 194 | 5.46 |
| 19d | 5/4/5 | 4 | 1.84 | 361 | 5.74 |
| 19e | 5/5/5 | 4 | 3.60 | 185 | 5.44 |
| 19f | 5/6/5 | 4 | 0.925 | 719 | 6.03 |

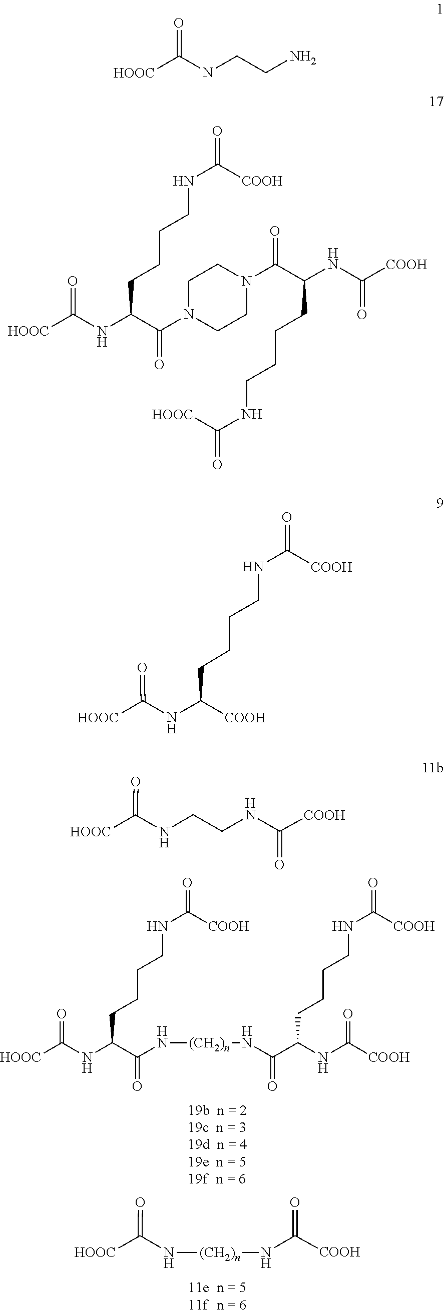

All references cited herein are incorporated herein by reference in their entireties. It will be appreciated by persons skilled in the art that the present invention is not limited to what has been particularly shown and described. Rather, the scope of the present invention is defined by the claims which follow. It should further be understood that the above description is only representative of illustrative examples of embodiments. The description has not attempted to exhaustively enumerate all possible variations. The alternate embodiments may not have been presented for a specific portion of the invention, and may result from a different combination of described portions, or that other un-described alternate embodiments may be available for a portion, is not to be considered a disclaimer of those alternate embodiments. It will be appreciated that many of those un-described embodiments are within the literal scope of the following claims, and others are equivalent.

We claim:

1-15. (canceled)

16. A compound of Formula (I), or a pharmaceutically acceptable salt thereof, 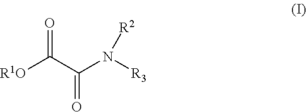
wherein, R¹ is H or C₁₋₈ alkyl;
R² is L-[NR^{2′}C(O)COOR^{1′}]ₘ;
L is a linker comprising one or more moieties selected from: 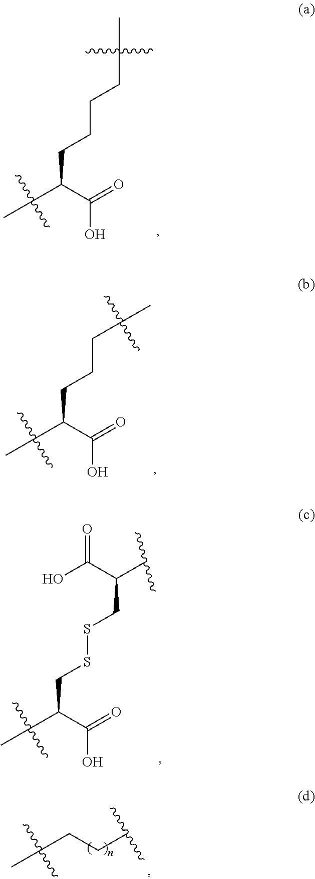
wherein n is 1, 2, 3, 4, 5, or 6, 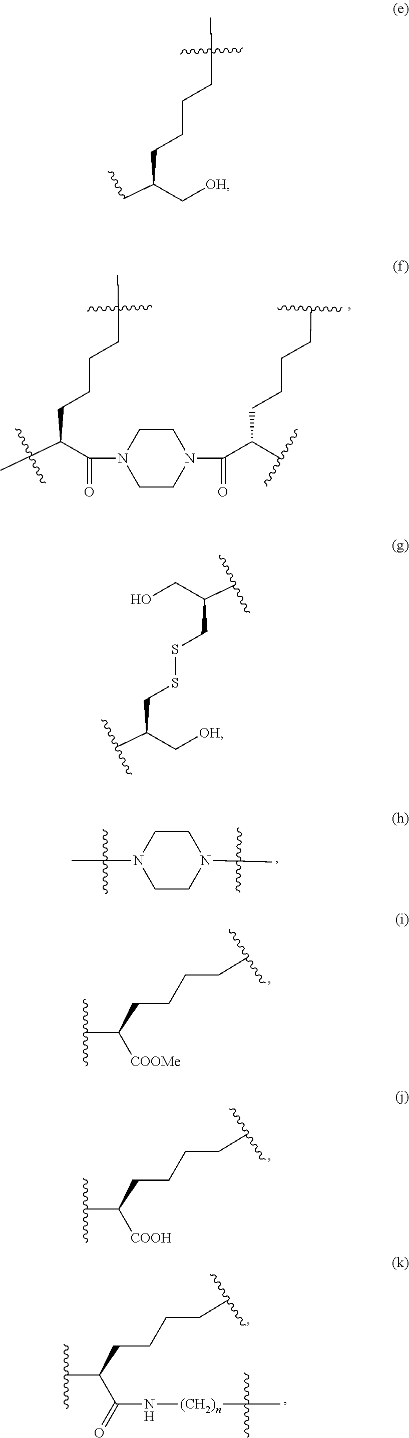
wherein n is 2, 3, 4, 5, or 6, 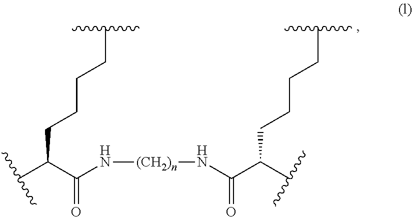
wherein n is 2, 3, 4, 5, 6, 7, or 8, 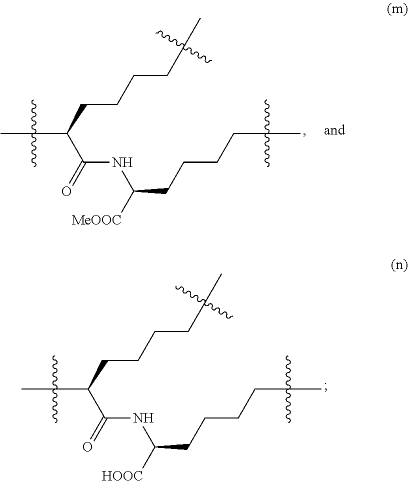 [SMILES: *CCCC[C@H](NC(=O)[C@@H](*)CCCC*)C(=O)OC ; *CCCC[C@H](NC(=O)[C@@H](*)CCCC*)C(=O)O]
m is 2 or 3;
each R^{1′} is independently H or C₁₋₈ alkyl;
each R^{2′} is independently H or C₁₋₄ alkyl; and
R³ is H or C₁₋₈ alkyl.

17. The compound of claim 16, or the pharmaceutically acceptable salt thereof, wherein
L is selected from: 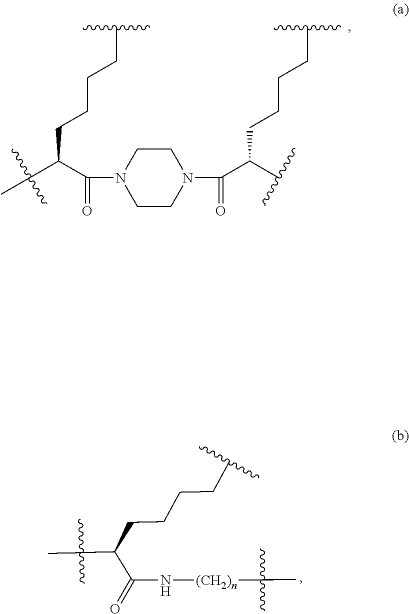
wherein n is 2, 3, 4, 5, or 6, 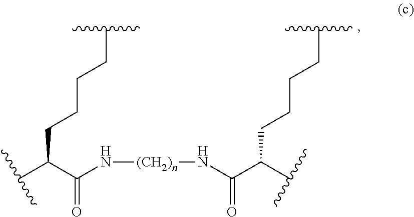
wherein n is 2, 3, 4, 5, 6, 7, or 8,

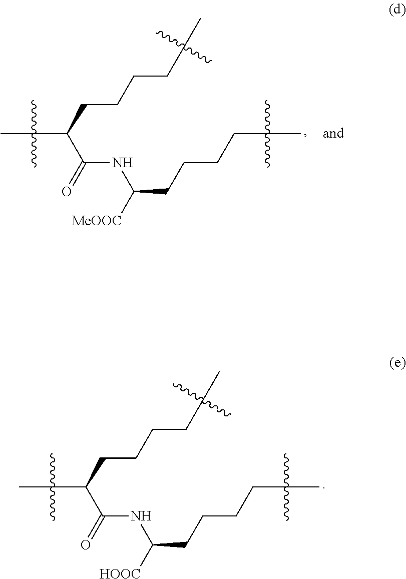 [SMILES: *CCCC[C@H](NC(=O)[C@@H](*)CCCC*)C(=O)OC ; *CCCC[C@H](NC(=O)[C@@H](*)CCCC*)C(=O)O]

18. The compound of claim 16, or the pharmaceutically acceptable salt thereof, wherein R³ is H or methyl.

19. The compound of claim 16, or the pharmaceutically acceptable salt thereof, wherein R¹ and R³ are each independently C₁₋₄ alkyl.

20. The compound of claim 16, or the pharmaceutically acceptable salt thereof, wherein R^{1′} and R^{2′} are each independently C₁₋₄ alkyl.

21. The compound of claim 16, or the pharmaceutically acceptable salt thereof, wherein m is 3.

22. The compound of claim 16, or the pharmaceutically acceptable salt thereof, wherein R³ and R^{2′} are both hydrogen.

23. The compound of claim 16, or the pharmaceutically acceptable salt thereof, wherein the compound is selected from:

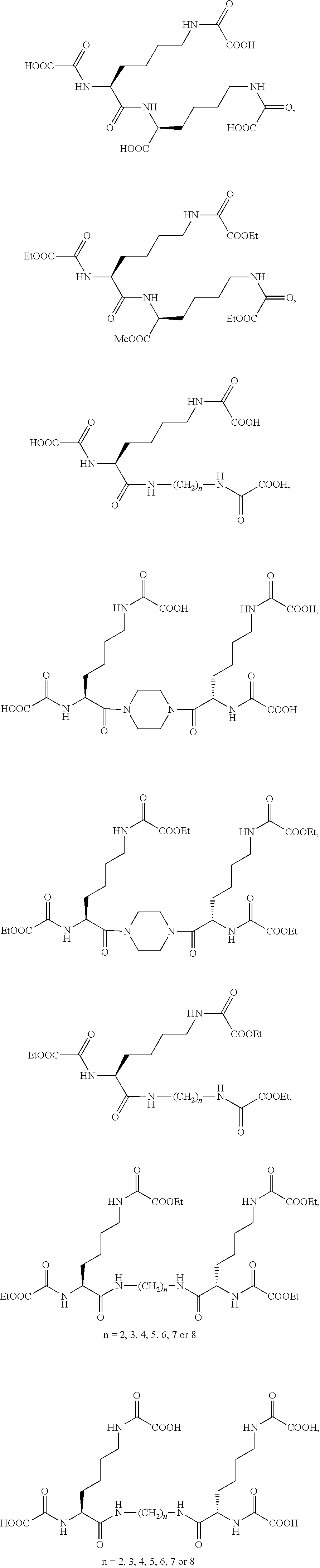

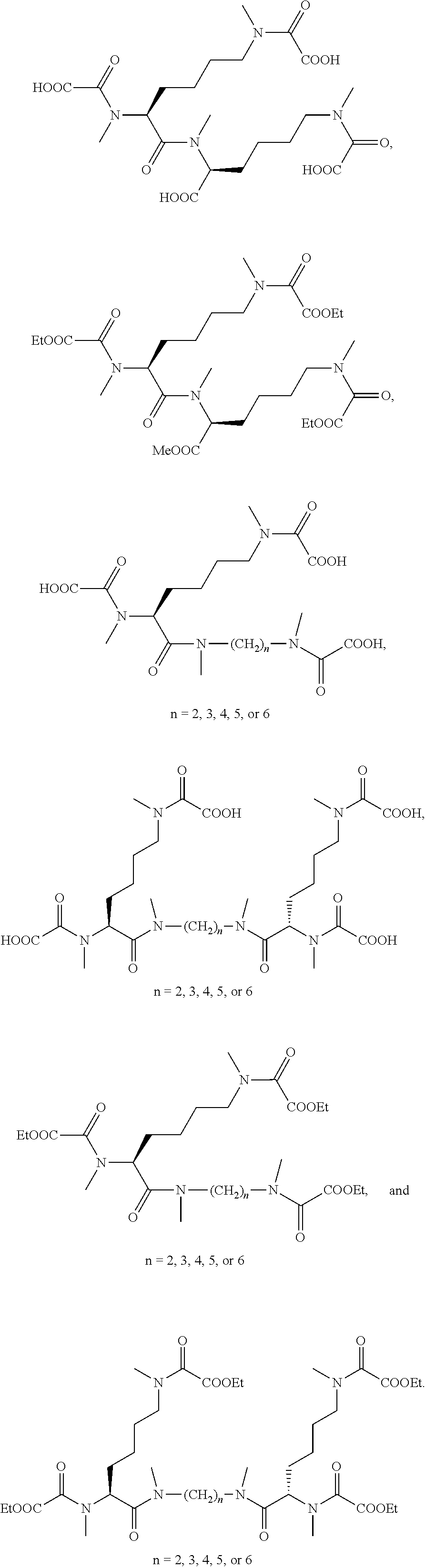

24. The compound of claim 16, or the pharmaceutically acceptable salt thereof, wherein the compound is selected from:

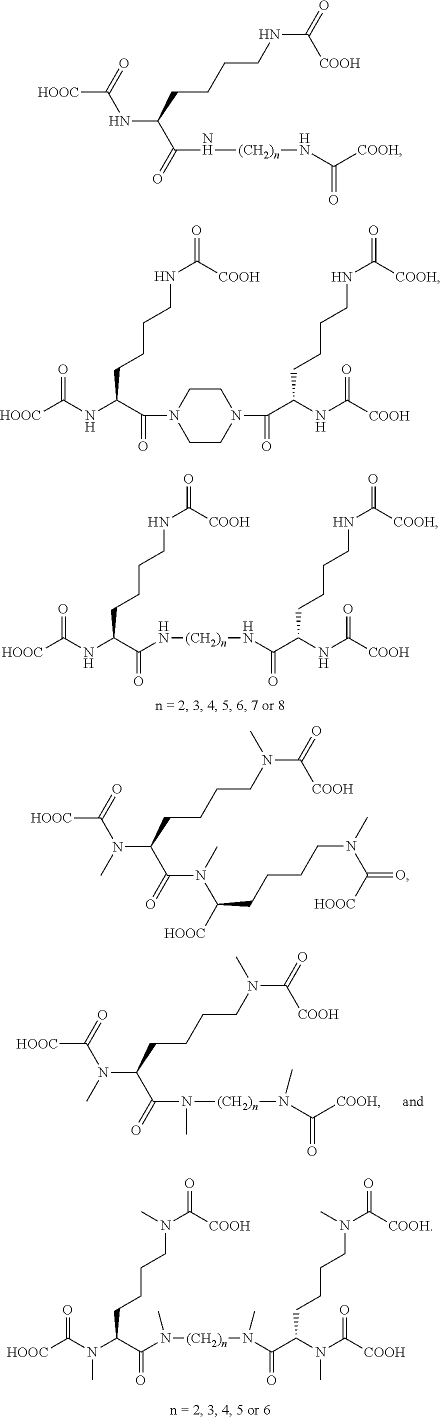

25. The compound of claim 16, or the pharmaceutically acceptable salt thereof, wherein the compound is selected from:

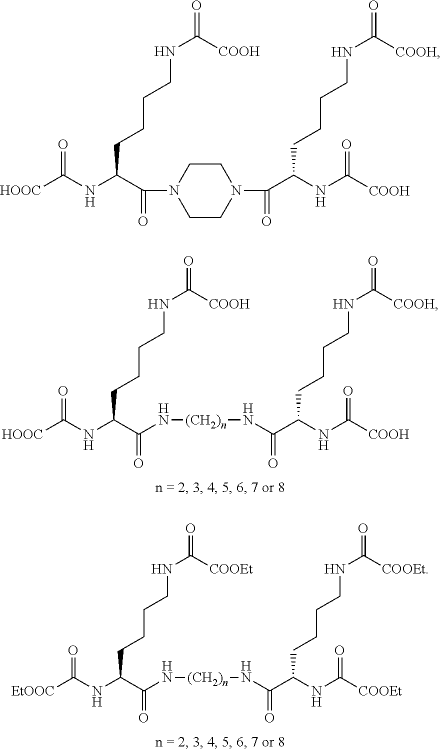

26. The compound of claim 16, or the pharmaceutically acceptable salt thereof, wherein the compound is:

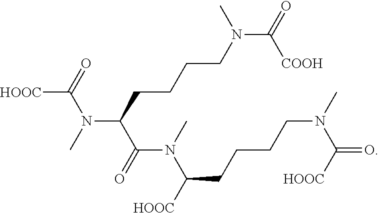 [SMILES: CN(CCCC[C@@H](C(=O)N(C)[C@@H](CCCCN(C)C(=O)C(=O)O)C(=O)O)N(C)C(=O)C(=O)O)C(=O)C(=O)O]

27. The compound of claim 16, or the pharmaceutically acceptable salt thereof, wherein the compound is:

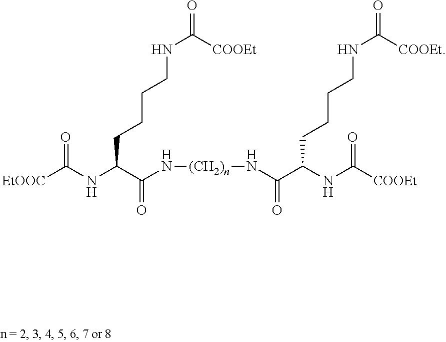

28. The compound of claim 16, or the pharmaceutically acceptable salt thereof, wherein the compound is:

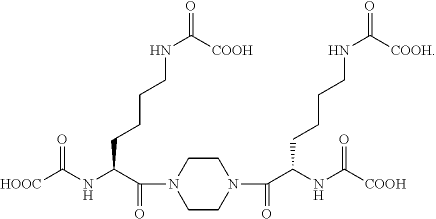 [SMILES: O=C(O)C(=O)NCCCC[C@H](NC(=O)C(=O)O)C(=O)N1CCN(C(=O)[C@H](CCCCNC(=O)C(=O)O)NC(=O)C(=O)O)CC1]

29. The compound of claim 16, or the pharmaceutically acceptable salt thereof, wherein the compound is:

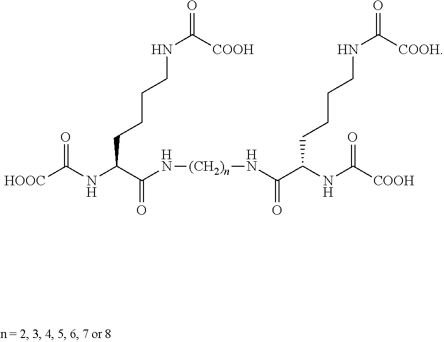

30. The compound of claim 29, or the pharmaceutically acceptable salt thereof, wherein n is 2, 3, or 4.

31. A pharmaceutical composition comprising a therapeutically effective amount of the compound of claim 16, or the pharmaceutically acceptable salt thereof.

32. A method of inhibiting formation of calcium oxalate crystals, comprising administering to a subject in need thereof a therapeutically effective amount of the compound of claim 16, or the pharmaceutically acceptable salt thereof.

33. The method of claim 32, wherein the subject has been diagnosed with a disease selected from the group consisting of urolithiasis, kidney disease, hypertension, osteoporosis, obesity, and oxalosis.

34. A compound, or a pharmaceutically acceptable salt thereof, selected from:

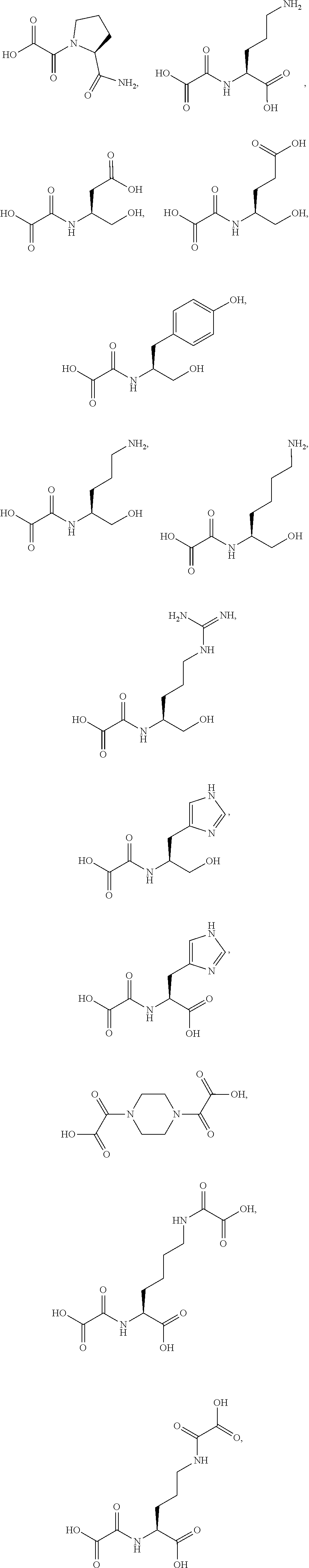

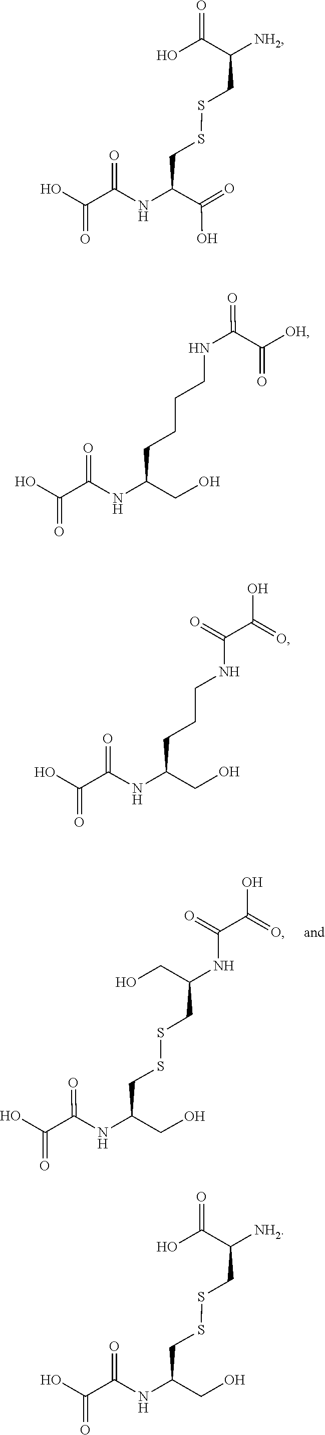 [SMILES: N[C@@H](CSSC[C@H](NC(=O)C(=O)O)C(=O)O)C(=O)O ; O=C(O)C(=O)NCCCC[C@@H](CO)NC(=O)C(=O)O ; O=C(O)C(=O)NCCC[C@@H](CO)NC(=O)C(=O)O ; O=C(O)C(=O)N[C@H](CO)CSSC[C@@H](CO)NC(=O)C(=O)O ; "and" ; N[C@@H](CSSC[C@@H](CO)NC(=O)C(=O)O)C(=O)O]

35. A pharmaceutical composition comprising a therapeutically effective amount of the compound of claim 34, or the pharmaceutically acceptable salt thereof.

36. A method of inhibiting formation of calcium oxalate crystals, comprising administering to a subject in need thereof a therapeutically effective amount of the compound of claim 34, or the pharmaceutically acceptable salt thereof.

37. The method of claim 36, wherein the subject has been diagnosed with a disease selected from the group consisting of urolithiasis, kidney disease, hypertension, osteoporosis, obesity, and oxalosis.

38. A method of inhibiting formation of calcium oxalate crystals, comprising administering to a subject in need thereof a therapeutically effective amount of a compound of Formula (I′), or a pharmaceutically acceptable salt thereof, 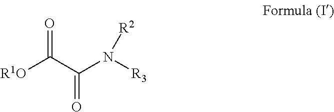
wherein
R¹ is H or C₁₋₈ alkyl;
R² is L-[NR^{2′}C(O)COOR^{1′}]ₘ;
L is a linker, wherein the linker comprises one or more moieties selected from an amino acid or moiety derived from an amino acid, C₁₋₈ alkylene, S—S, —NH—C₁₋₈ alkylene, —NH—C₁₋₈ alkyleneN(H)—, optionally substituted phenyl, optionally substituted 5-10 membered heteroaryl, 5-10 membered carbocyclic, and 5-10 membered heterocyclic, wherein when L is optionally substituted, the optional substituents are selected from one or more moieties selected from COOH, COOC₁₋₆ alkyl, halogen, halo-C₁₋₆ alkyl, C₁₋₄ alkylene-OH, and CN;
m is 2 or 3,
each R^{1′} is independently H or C₁₋₈ alkyl;
each R^{2′} is independently H or C₁₋₄ alkyl;
R³ is H or C₁₋₈ alkyl;
or R² and R³ together with the nitrogen to which they are attached form a 5-8 membered heterocyclic ring, wherein the 5-8 membered heterocyclic ring is optionally substituted with C₁₋₈ alkyl, COOH, COOC₁₋₈ alkyl, C(O)NR^{a}R^{b}, or C(O)COOR^{1′}, and
R^{a} and R^{b} are independently H or C₁₋₈ alkyl.
